(19) United States
(12) Patent Application Publication
(10) Pub. No.: US 2024/0212792 A1
(43) Pub. Date: Jun. 27, 2024

(54) RNA-PROTEIN INTERACTION PREDICTION METHOD AND DEVICE, STORAGE MEDIUM AND ELECTRONIC DEVICE

(71) Applicant: BOE Technology Group Co., Ltd., Beijing (CN)

(72) Inventors: Sifan WANG, Beijing (CN); Zhenzhong ZHANG, Beijing (CN)

(73) Assignee: BOE Technology Group Co., Ltd., Beijing (CN)

(21) Appl. No.: 17/916,540

(22) PCT Filed: Sep. 27, 2021
(86) PCT No.: PCT/CN2021/121103

§ 371 Date: Sep. 30, 2022

(51) Int. Cl. G16B 40/20; G06N 5/022

(52) U.S. Cl. CPC G16B 40/20; G06N 5/022

(57) ABSTRACT

An RNA-protein interaction prediction method and device, a medium and an electronic device are provided. The method includes: obtaining an RNA-protein pair to be predicted; performing feature extraction on the RNA-protein pair to obtain sequence features of the RNA-protein pair; vectorizing the RNA-protein pair to obtain an RNA sequence representation vector and a protein sequence representation vector in the RNA-protein pair; based on the sequence features of the RNA-protein pair, the RNA sequence representation vector and the protein sequence representation vector in the RNA-protein pair, obtaining at least one predicted interaction value of the RNA-protein pair using at least one interaction prediction model; and determining interaction between the RNA and the protein according to the at least one predicted interaction value.

### TECHNICAL FIELD

The present disclosure relates to the artificial intelligence technical field, and in particular, to an RNA-protein interaction prediction method, an RNA-protein interaction prediction device, a computer-readable storage medium and an electronic device.

### BACKGROUND

Noncoding RNA (ncRNA) is involved in many complex cellular processes, plays an important role in life processes such as alternative splicing, chromatin modification and epigenetics, and is closely related to many diseases. Studies have shown that most non-coding RNAs achieve their regulatory functions by interacting with proteins. Therefore, studying the interaction between non-coding RNA and protein is of great significance for revealing the molecular mechanism of non-coding RNA in human diseases and life activities, and has become one of the important ways to analyze the function of non-coding RNA and protein.

It should be noted that the information disclosed in the background section above is only used to enhance the understanding of the background of the present disclosure, and therefore may include information that does not constitute the prior art known to those skilled in the art.

### SUMMARY

The present disclosure provides an RNA-protein interaction prediction method, an RNA-protein interaction prediction device, a computer-readable storage medium and an electronic device.

An embodiment of the present disclosure provides an RNA-protein interaction prediction method, including:
obtaining an RNA-protein pair to be predicted;
performing feature extraction on the RNA-protein pair to be predicted to obtain sequence features of the RNA-protein pair to be predicted;
vectorizing the RNA-protein pair to be predicted to obtain an RNA sequence representation vector and a protein sequence representation vector in the RNA-protein pair to be predicted;
based on the sequence features of the RNA-protein pair to be predicted, the RNA sequence representation vector and the protein sequence representation vector in the RNA-protein pair to be predicted, obtaining at least one predicted interaction value of the RNA-protein pair to be predicted using at least one interaction prediction model; and
determining interaction between the RNA and the protein according to the at least one predicted interaction value.

In an example embodiment of the present disclosure, performing the feature extraction on the RNA-protein pair to be predicted to obtain the sequence features of the RNA-protein pair to be predicted includes:
obtaining an original sequence feature set; and
determining the sequence features of the RNA-protein pair to be predicted according to the original sequence feature set.

In an example embodiment of the present disclosure, determining the sequence features of the RNA-protein pair to be predicted according to the original sequence feature set includes:
converting an RNA sequence and a protein sequence in the RNA-protein pair to be predicted into k-mer subsequences, respectively; and
searching each of the k-mer subsequences in the original sequence feature set, and obtaining the sequence features of the RNA-protein pair to be predicted according to a search result.

In an example embodiment of the present disclosure, determining the sequence features of the RNA-protein pair to be predicted according to the original sequence feature set includes:
converting an RNA sequence and a protein sequence in the RNA-protein pair to be predicted into k-mer subsequences, respectively, wherein the k-mer subsequences includes RNA k-mer subsequences and protein k-mer subsequences;
combining the RNA k-mer subsequences and the protein k-mer subsequences to obtain a plurality of RNA-protein k-mer subsequence pairs; and
searching each of the plurality of RNA-protein k-mer subsequence pairs in the original sequence feature set, and obtaining the sequence features of the RNA-protein pair to be predicted according to a search result.

In an example embodiment of the present disclosure, determining the sequence features of the RNA-protein pair to be predicted according to the original sequence feature set includes:
converting an RNA sequence and a protein sequence in the RNA-protein pair to be predicted into k-mer subsequences, respectively, wherein the k-mer subsequences include RNA k-mer subsequences and protein k-mer subsequences;
searching each of the k-mer subsequences in the original sequence feature set to obtain first sequence features;
combining the RNA k-mer subsequences and the protein k-mer subsequences to obtain a plurality of RNA-protein k-mer subsequence pairs;
searching each of the plurality of RNA-protein k-mer subsequence pairs in the original sequence feature set to obtain second sequence features; and
constituting the sequence features of the RNA-protein pair to be predicted by the first sequence features and the second sequence features.

In an example embodiment of the present disclosure, vectorizing the RNA-protein pair to be predicted to obtain the RNA sequence representation vector and the protein sequence representation vector in the RNA-protein pair to be predicted includes:
converting an RNA sequence and a protein sequence in the RNA-protein pair to be predicted into k-mer subsequences, respectively, wherein the k-mer subsequences include M RNA k-mer subsequences and N protein k-mer subsequences;
vectorizing each of the RNA k-mer subsequences to obtain M RNA k-mer vectors;
concatenating the M RNA k-mer vectors to obtain the RNA sequence representation vector;
vectorizing each of the protein k-mer sequences to obtain N protein k-mer vectors; and
concatenating the N protein k-mer vectors to obtain the protein sequence representation vector.

In an example embodiment of the present disclosure, vectorizing the RNA-protein pair to be predicted to obtain the RNA sequence representation vector and the protein sequence representation vector in the RNA-protein pair to be predicted includes:
vectorizing each base included in an RNA sequence in the RNA-protein pair to be predicted to obtain a plurality of base vectors;
concatenating the plurality of base vectors to obtain the RNA sequence representation vector;
vectorizing each amino acid vector included in a protein sequence in the RNA-protein pair to be predicted to obtain a plurality of amino acid vectors; and
concatenating the plurality of amino acid vectors to obtain the protein sequence representation vector.

In an example embodiment of the present disclosure, based on the sequence features of the RNA-protein pair to be predicted, the RNA sequence representation vector and the protein sequence representation vector in the RNA-protein pair to be predicted, obtaining the at least one predicted interaction value of the RNA-protein pair to be predicted using the at least one interaction prediction model includes:
based on the sequence features of the RNA-protein pair to be predicted, the RNA sequence representation vector and the protein sequence representation vector in the RNA-protein pair to be predicted, obtaining a plurality of predicted interaction values of the RNA-protein pair to be predicted using at least three interaction prediction models.

In an example embodiment of the present disclosure, based on the sequence features of the RNA-protein pair to be predicted, the RNA sequence representation vector and the protein sequence representation vector in the RNA-protein pair to be predicted, obtaining the at least one predicted interaction value of the RNA-protein pair to be predicted using the at least one interaction prediction model includes:
inputting the sequence features of the RNA-protein pair to be predicted into a first interaction prediction model to obtain a first predicted interaction value; and
inputting the RNA sequence representation vector and the protein sequence representation vector in the RNA-protein pair to be predicted into a second interaction prediction model to obtain a second predicted interaction value;
wherein at least one first interaction model and at least two second interaction prediction models are included; or, at least two first interaction models and at least one second interaction prediction model are included.

In an example embodiment of the present disclosure, based on the sequence features of the RNA-protein pair to be predicted, the RNA sequence representation vector and the protein sequence representation vector in the RNA-protein pair to be predicted, obtaining a plurality of predicted interaction values of the RNA-protein pair to be predicted using the at least one interaction prediction model includes:
inputting the sequence features of the RNA-protein pair to be predicted into a traditional machine learning model to obtain a first predicted interaction value; and
inputting the RNA sequence representation vector and the protein sequence representation vector in the RNA-protein pair to be predicted into a deep learning model to obtain a second predicted interaction value;
wherein at least one traditional machine learning model and at least two deep learning models are included; or, at least two traditional machine learning models and at least one deep learning models are included.

In an example embodiment of the present disclosure, the traditional machine learning model includes at least one of a support vector machine model, a logistic regression model and a decision tree model, and the deep learning model includes at least one of a convolutional neural network model and a recurrent neural network model.

In an example embodiment of the present disclosure, determining the interaction between the RNA and the protein according to the at least one predicted interaction value includes:
marking a plurality of predicated interaction values to obtain a plurality of marker values; and
summing the plurality of marker values, and determining the interaction between the RNA and the protein according to a sum result.

In an example embodiment of the present disclosure, obtaining the original sequence feature set includes:
obtaining an original data set;
performing feature extraction on each RNA-protein pair in the original data set to obtain the original sequence feature set.

In an example embodiment of the present disclosure, performing the feature extraction on each RNA-protein pair in the original data set to obtain the original sequence feature set includes:
performing permutation and combination on basic units of the RNA and the protein respectively to obtain k-mer subsequences;
calculating an average value of the number of occurrences of each k-mer subsequence in each RNA-protein pair, and calculating a variance of each k-mer subsequence according to the average value of the number of occurrences; and
determining the original sequence feature set according to a magnitude of the variance of each k-mer subsequence.

In an example embodiment of the present disclosure, calculating the average value of the number of occurrences of each k-mer subsequence in each RNA-protein pair, and calculating the variance of each k-mer subsequence according to the average value of the number of occurrences includes:
traversing the original data set to determine the number of occurrences of each k-mer subsequence in each RNA-protein pair;
counting the number of occurrences of each k-mer subsequence in each RNA-protein pair to obtain a total number of occurrences of each k-mer subsequence in the original data set;
calculating the average value of the number of occurrences of each K-mer subsequence in each RNA-protein pair according to the total number of occurrences; and
calculating the variance of each k-mer subsequence according to the average value of the number of occurrences of each k-mer subsequence in each RNA-protein pair and the number of occurrences of each k-mer subsequence in each RNA-protein pair.

In an example embodiment of the present disclosure, calculating the variance of each k-mer subsequence according to the average value of the number of occurrences of each k-mer subsequence in each RNA-protein pair and the number of occurrences of each k-mer subsequence in each RNA-protein pair includes:
calculating the variance s² of each k-mer subsequence according to:

$${s}^{2} = \frac{{( m - {x}_{1} )}^{2} + {( m - {x}_{2} )}^{2} + … + {( m - {x}_{n} )}^{2}}{n}$$

where n is the number of RNA-protein pairs in the original data set, m is the average value of the number of occurrences of each k-mer subsequence in each RNA-protein pair, and xₙ is the number of occurrence of each k-mer subsequence in an n-th RNA-protein pair.

In an example embodiment of the present disclosure, determining the original sequence feature set according to the magnitude of the variance of each k-mer subsequence includes:
determining k-mer subsequences which meet a preset condition according to the variance of each k-mer subsequence, and constituting the original sequence feature set by the k-mer subsequences which meet the preset condition.

In an example embodiment of the present disclosure, performing feature extraction on each RNA-protein pair in the original data set to obtain the original sequence feature set includes:
converting an RNA sequence and a protein sequence in each RNA-protein pair into k-mer subsequences respectively to obtain k-mer subsequence pairs; and
counting a relative frequency of occurrence of each of the k-mer subsequence pairs in the original data set, and constituting the original sequence feature set by k-mer subsequence pairs which meet a preset condition for relative frequency of occurrence.

In an example embodiment of the present disclosure, the method further includes:
training the at least one interaction prediction model.

In an example embodiment of the present disclosure, training the at least one interaction prediction model includes:
obtaining a training data set, wherein the training data set includes positive RNA-protein pairs and negative RNA-protein pairs;
with the training data set as an input of the at least one interaction prediction model, iteratively updating model parameters of the at least one interaction prediction model, and when an iteration termination condition is met, completing training of all model parameters so as to predict the interaction of the RNA-protein pair to be predicted using the trained at least one interaction prediction model.

In an example embodiment of the present disclosure, the method further includes:
outputting a prediction result of the interaction between the RNA and the protein.

Another embodiment of the present disclosure provides an RNA-protein interaction prediction device, including:
a data obtaining module configured to obtain an RNA-protein pair to be predicted;
a feature extraction module configured to perform feature extraction on the RNA-protein pair to be predicted to obtain sequence features of the RNA-protein pair to be predicted;
a data vectorization module configured to vectorize the RNA-protein pair to be predicted to obtain an RNA sequence representation vector and a protein sequence representation vector in the RNA-protein pair to be predicted;
an interaction prediction module configured to, based on the sequence features of the RNA-protein pair to be predicted, the RNA sequence representation vector and the protein sequence representation vector in the RNA-protein pair to be predicted, obtain at least one predicted interaction value of the RNA-protein pair to be predicted using at least one interaction prediction model; and
an interaction determination module configured to determine interaction between the RNA and the protein according to the at least one predicted interaction value.

Another embodiment of the present disclosure provides a computer-readable storage medium on which a computer program is stored, wherein when the computer program is executed by a processor, the processor is caused to perform the method according to any one of the above embodiments.

An embodiment of the present disclosure provides an electronic device, including:
a processor; and
a memory for storing executable instructions which are executable by the processor;
wherein when the executable instructions are executed by the processor, the processor is configured to perform the method according to any one of the above embodiments.

It is to be understood that the foregoing general description and the following detailed description are exemplary and explanatory only and are not restrictive of the present disclosure.

### BRIEF DESCRIPTION OF THE DRAWINGS

The accompanying drawing, which are incorporated herein and form a part of the specification, illustrate the embodiments of the present disclosure and, together with the description, further serve to explain the principles of the embodiments of the present disclosure. Obviously, the drawings in the following description are only some embodiments of the present disclosure. For those of ordinary skill in the art, other drawings can be obtained based on these drawings without creative work.

FIG. 1 shows a schematic diagram of an example system architecture in which an RNA-protein interaction prediction method and device according to embodiments of the present disclosure can be applied.

FIG. 2 schematically shows a flow chart of an RNA-protein interaction prediction method according to an embodiment of the present disclosure.

FIG. 3 schematically shows a flow chart of determining sequence features of an RNA-protein pair to be predicted according to an embodiment of the present disclosure.

FIG. 4 schematically shows a flow chart of obtain an original sequence feature set according to an embodiment of the present disclosure.

FIG. 5 schematically shows a flow chart of training an interaction prediction model according to an embodiment of the present disclosure.

FIG. 6 schematically shows a block diagram of an RNA-protein interaction prediction device according to an embodiment of the present disclosure.

FIG. 7 shows a schematic structural diagram of a computer system suitable for implementing an electronic device according to an embodiment of the present disclosure.

### DETAILED DESCRIPTION

Example embodiments will now be described more fully with reference to the accompanying drawings. However, the example embodiments can be implemented in various forms and should not be construed as limited to the examples set forth herein; rather, providing these embodiments makes the present disclosure more comprehensive and complete, and conveys the concepts of the example embodiments comprehensively to those skilled in the art. The described features, structures, or characteristics may be combined in any suitable manner in one or more embodiments. In the following description, numerous specific details are provided in order to give a thorough understanding of the embodiments of the present disclosure. However, those skilled in the art will appreciate that the technical solutions of the present disclosure may be practiced without one or more of the specific details, or other methods, components, devices, steps, etc. may be employed. In other instances, well-known solutions have not been shown or described in detail to avoid obscuring aspects of the present disclosure.

In addition, the drawings are merely schematic illustrations of the present disclosure and are not necessarily drawn to scale. The same reference numerals in the drawings represent the same or similar parts, and thus repeated descriptions thereof will be omitted. Some block diagrams shown in the drawings are functional entities and do not necessarily correspond to physically or logically independent entities. These functional entities may be implemented in the form of software, or implemented in one or more hardware modules or integrated circuits, or implemented in different networks and/or processor devices and/or microcontroller devices.

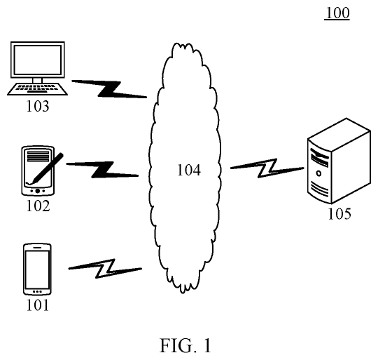

FIG. 1 shows a schematic diagram of a system architecture of an example application environment to which an RNA-protein interaction prediction method and device according to an embodiment of the present disclosure can be applied.

As shown in FIG. 1, the system architecture 100 may include one or more of terminal devices 101, 102, 103, a network 104 and a server 105. The network 104 is a medium used to provide a communication link between the terminal devices 101, 102, 103 and the server 105. The network 104 may include various connection types, such as wired, wireless communication links, or fiber optic cables, and so on. The terminal devices 101, 102, and 103 may be various electronic devices, including but not limited to desktop computers, portable computers, smart phones, and tablet computers and so on. It should be understood that the numbers of terminal devices, networks and servers in FIG. 1 are merely illustrative. There can be any number of terminal devices, networks and servers according to implementation needs. For example, the server 105 may be a server, a server cluster composed of multiple servers, or a cloud computing platform or a virtualized center. Specifically, the server 105 may be configured to perform: obtaining an RNA-protein pair to be predicted; performing feature extraction on the RNA-protein pair to obtain sequence features of the RNA-protein pair to be predicted; vectorizing the RNA-protein pair to be predicted to obtain an RNA sequence representation vector and a protein sequence representation vector in the RNA-protein pair to be predicted; based on the sequence features of the RNA-protein pair to be predicted, the RNA sequence representation vector and the protein sequence representation vector in the RNA-protein pair to be predicted, obtaining at least one predicted interaction value of the RNA-protein pair using at least one interaction prediction model; and determining interaction between the RNA and the protein according to the at least one predicted interaction value.

The RNA-protein interaction prediction method provided by the embodiments of the present disclosure is generally performed by the server 105. Accordingly, the RNA-protein interaction prediction device is generally set in the server 105, and the server can send the prediction result of the interaction of the RNA-protein to be predicted to a terminal device which may display the prediction result to a user. However, it should be easily understood by those skilled in the art that the RNA-protein interaction prediction method provided by the embodiments of the present disclosure can alternatively be performed by one or more of the terminal devices 101, 102, and 103. Correspondingly, the RNA-protein interaction prediction device can also be set in one or more of the terminal devices 101, 102, and 103. For example, a terminal device performs the prediction method, the prediction result can be directly displayed on the display screen of the terminal device, or the prediction result can be provided to the user by means of voice broadcast, and embodiments of the present disclosure do not impose specific limitations on this.

The technical solutions of the embodiments of the present disclosure will be described in detail below.

Currently, noncoding RNA-protein interactions (ncRPIs) can be studied using experimental methods. Traditional experimental methods can obtain valuable data experimentally to construct ncRNA-protein interaction networks, but these methods are expensive and time-consuming.

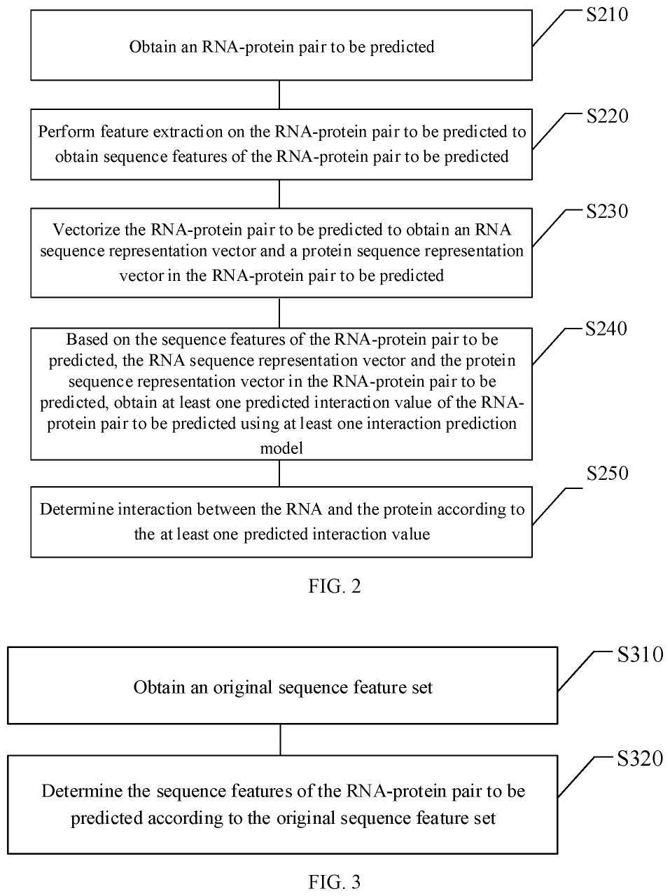

An example embodiment of the present disclosure provides an RNA-protein interaction prediction method. The method may be applied to the foregoing server 105 or one or more of the foregoing terminal devices 101, 102, and 103, and this embodiment does not impose specific limitations on this. Referring to FIG. 2, the RNA-protein interaction prediction method may include the following steps S210 to S250.

In step S210, an RNA-protein pair to be predicted is obtained.

In step S220, feature extraction is performed on the RNA-protein pair to be predicted to obtain sequence features of the RNA-protein pair to be predicted.

In step S230, the RNA-protein pair to be predicted is vectorized to obtain an RNA sequence representation vector and a protein sequence representation vector in the RNA-protein pair to be predicted.

In step S240, based on the sequence features of the RNA-protein pair to be predicted, the RNA sequence representation vector and the protein sequence representation vector in the RNA-protein pair to be predicted, at least one predicted interaction value of the RNA-protein pair to be predicted is obtained using at least one interaction prediction model.

In step S250, interaction between the RNA and the protein is determined according to the at least one predicted interaction value.

In the RNA-protein interaction prediction method provided by the example embodiment of the present disclosure, the RNA-protein pair to be predicted is obtained; feature extraction is performed on the RNA-protein pair to be predicted to obtain sequence features of the RNA-protein pair to be predicted; the RNA-protein pair to be predicted is vectorized to obtain the RNA sequence representation vector and the protein sequence representation vector in the RNA-protein pair to be predicted; based on the sequence features of the RNA-protein pair to be predicted, the RNA sequence representation vector and the protein sequence representation vector in the RNA-protein pair to be predicted, at least one predicted interaction value of the RNA-protein pair to be predicted is obtained using at least one interaction prediction model; and interaction between the RNA and the protein is determined according to the at least one predicted interaction value.

On the one hand, through feature extraction and vectorization of the RNA-protein pair, the association between the RNA sequence and the protein sequence can be fully mined, so as to accurately predict the interaction between RNA and protein. On the other hand, characteristics of the interaction prediction models may be effectively combined to further improve the accuracy in RNA-protein interaction prediction.

Hereinafter, the above steps of the example embodiment will be described in more detail.

In step S210, the RNA-protein pair to be predicted is obtained.

In an example embodiment, at least one RNA-protein pair to be predicted may be obtained. The interaction between an RNA and a protein in each RNA-protein pair to be predicted is unknown. Exemplarily, a user can input the RNA-protein pair to be predicted through a terminal device. For example, the user can manually input the RNA-protein pair to be predicted, or can input the RNA-protein pair to be predicted by voice, which is not specifically limited in this example. For example, an RNA can be input, and a protein can be input, and the order of input of the two is not limited. For example, RNA and protein can be entered into different text boxes or into the same text box. For example, after the input is completed, a “start prediction” button may be clicked/tapped to start executing the prediction steps provided in some embodiments of the present application.

The interaction between an RNA and a protein means that the function of the protein is reflected in the interactions with other proteins and RNA. For example, protein-RNA interactions play an important role in protein synthesis. Meanwhile, the performance of many functions of RNA is also inseparable from the interaction with proteins. The interaction can be regulation, guidance, etc., which is not limited here. For example, in the presence of interactions, the RNA can guide the synthesis of the protein, or the RNA can regulate the function of the protein. The interaction between an RNA and a protein can also refer to that the two can regulate each other's life cycle and functions through physical interaction. Illustratively, RNA coding sequences can direct the synthesis of proteins, and correspondingly, proteins can also regulate RNA expression and functions.

After obtaining the RNA-protein pair to be predicted, multiple interaction prediction models can be used to predict the interaction of each input RNA-protein pair to be predicted, and where there is interaction may be determined for each to-be-predicted RNA-protein pair according to the prediction result. Meanwhile, the prediction result of the RNA-protein pair interaction to be predicted may also be output to a terminal device for a user to view. For example, the prediction result may be directly displayed on the display screen of the terminal device, or the prediction result may be provided to the user by means of voice broadcast, which is not specifically limited in this example.

In other examples, at least one RNA sequence to be predicted may be obtained, and the interaction prediction model is used to search a database to find at least one protein sequence that interact with each input RNA sequence to be predicted. Exemplarily, after the user inputs the RNA sequence to be predicted through the terminal device, at least one protein sequence in the database may be selected, and multiple RNA-protein pairs may be composed of the RNA sequence to be predicted and the protein sequences. Then, the interaction prediction model may be used to predict the interaction of each RNA-protein pair, and a protein sequence that can interact with the RNA sequence to be predicted may be output according to the prediction result. Preferably, several kinds of protein sequences may be pre-stored in the database for easy recall when predicting RNA-protein pair interactions. For example, the protein sequences may be stored in the Redis database or in the MySQL database, and then the protein sequences to be predicted can be queried and selected in real time. Redis is a key-value storage system. The storage of the protein sequences in the Redis database may include: key-value pairs formed by sequence identifiers and corresponding protein sequences, where the key is the sequence identifiers, and the value is the corresponding protein sequences. As an efficient caching technology, Redis can support more than 100K+ read and write frequencies per second, and has certain advantages in data reading and storage speed. MySQL is an associative database management system. An associative database store data in different tables instead of storing all data uniformly, increasing storage speed and improving flexibility. MySQL has stable advantages in data storage and can avoid occurrence of data loss.

It will be appreciated that several kinds of RNA sequences may also be pre-stored in the database for easy recall when predicting the RNA-protein pair interactions. Therefore, it is also possible to obtain at least one protein sequence to be predicted, and search the database to find RNA sequences that interact with each input protein sequence to be predicted through the interaction prediction model. Similarly, after the user enters the protein sequence through the terminal device, at least one RNA sequence in the database may be selected, and multiple RNA-protein pairs are composed of the protein sequence to be predicted and at least one RNA sequence. Then, the interaction prediction model may be used to predict the interaction of each RNA-protein pair. At least one RNA sequence that can interact with the protein sequence to be predicted is output according to the prediction result, which is not specifically limited in the present disclosure.

In step S220, feature extraction is performed on the RNA-protein pair to be predicted to obtain sequence features of the RNA-protein pair to be predicted.

In an example embodiment, a case where at least one RNA-protein pair to be predicted is obtained and the interaction thereof is predicted is taken as an example. Before predicting the interaction of each RNA-protein pair to be predicted by the interaction prediction model, the input features of the interaction prediction model need to be obtained. Exemplarily, feature extraction may be performed on the RNA-protein pair to be predicted, that is, feature extraction is performed on the RNA sequence and the protein sequence in the RNA-protein pair to be predicted in turn to obtain corresponding RNA sequence features and protein sequence features. The sequence features of the RNA-protein pair to be predicted are composed of the RNA sequence features and the protein sequence features. The sequence features may be used as the input of the interaction prediction model. The RNA-protein pair to be predicted may be vectorized, that is, the RNA sequence and protein sequence in the RNA-protein pair are respectively vectorized to obtain a corresponding RNA sequence representation vector and a protein representation vector. The RNA sequence representation vector and the protein representation vector are used as input the input of the interaction prediction model. It is also possible to perform feature extraction and vectorization processing on the RNA-protein pair to be predicted at the same time to obtain the sequence features of the RNA-protein pair to be predicted, the RNA sequence representation vector and the protein representation vector in the RNA-protein pair to be predicted. The sequence features of the RNA-protein pair to be predicted, the RNA sequence representation vector and the protein representation vector in the RNA-protein pair to be predicted can all be used as the input of the interaction prediction model, which are not specifically limited in the present disclosure.

Referring to FIG. 3, feature extraction may be performed on the RNA-protein pair to be predicted according to steps S310 and S320.

In step S310, an original sequence feature set is obtained.

In an example embodiment, an original data set may be obtained, and feature extraction is performed on each RNA-protein pair in the original data set to obtain the original sequence feature set.

For example, the RPI1807 data set may be used as the original data set. The RPI1807 data set contains 3243 RNA-protein pairs. Among the 3243 RNA-protein pairs, 1807 positive cases and 1436 negative cases are included. A positive case may indicate that there is an interaction between the RNA and the protein in an RNA-protein pair, and a negative case may indicate that there is no interaction between the RNA and the protein in an RNA-protein pair. It can be understood that in other examples, the RPI2241 data set, the RPI369 data set, and so on may also be used as the original data set for experiments, which are not specifically limited in the present disclosure.

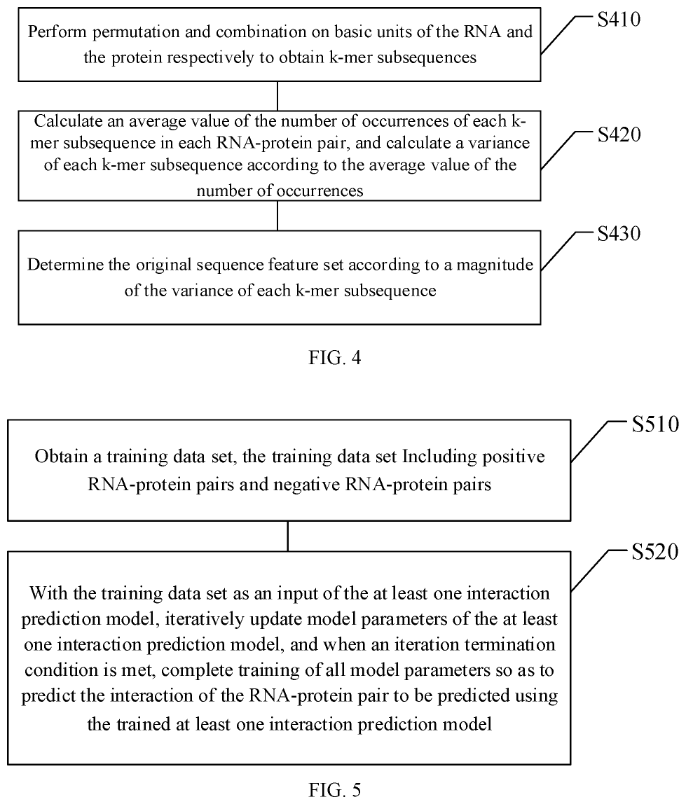

After obtaining the original data set, referring to FIG. 4, feature extraction may be performed on the RNA-protein pairs in the original data set according to steps S410 to S430 to obtain the original sequence data set.

In step S410, permutation and combination is performed on basic units of the RNA and the protein respectively to obtain k-mer subsequences.

For example, the base is the basic unit of an RNA. An RNA sequence may include four bases: namely, adenine (A), uracil (U), guanine (G) and cytosine (C). All k-mer subsequences of RNA sequences may be obtained by permutation and combination of the four bases. For example, amino acids are the basic units of proteins. For a protein sequence, 20 amino acids may be included, and the 20 amino acids may be encoded as A, G, V, I, L, F, P, Y, M, T, S, H, N, Q, W, R, K, D, E, C. Exemplarily, according to the physicochemical properties of amino acids, the 20 amino acids may be divided into {A, G, V}, {I, L, F, P}, {Y, M, T, S}, {H, N, Q, W}, {R, K}, {D, E} and {C}, a total of 7 types, and each type of amino acid is recoded, for example, they may be encoded as 1, 2, 3, 4, 5, 6 and 7. For example, the protein sequence ALQDVG may be converted to 124611. Then, all the k-mer subsequences of the amino acid sequences may be obtained by permuting and combining the 7 types of amino acids. In other examples, the 20 kinds of amino acids may also be classified according to the amino acid composition, and the k-mer subsequences of the amino acid sequences may be obtained by directly permuting and combining the 20 kinds of amino acids without classification, which is not specifically limited in this disclosure.

A k-mer subsequence refers to a k-complex consisting of k bases or k types of amino acids as a group. Correspondingly, in an example embodiment of the present disclosure, the k-mer subsequences may include RNA k-mer subsequences and protein k-mer subsequences. Exemplarily, a k-mer subsequence may refer to an RNA k-mer subsequence obtained by permuting and combining 4 kinds of bases, and for a certain k value, 4k kinds of k-mer subsequences can be obtained. A k-mer subsequence may also refer to a protein k-mer subsequence obtained by permuting and combining 7 types of amino acids. For a certain k value, 7k kinds of k-mer subsequences may be obtained. It can be understood that the classification of the 20 amino acids into 7 types is only illustrative, and the classification may not be performed. Similarly, the four bases of RNA sequences may also be classified according to actual needs.

In an example embodiment of the present disclosure, the value of k may be one or more, and the specific value of k may be adjusted according to the actual situation, which is not limited herein. In an example, k takes two values of 3 and 4 as an example for description. There are 4³=64 kinds of 3-mer subsequences and 44=256 kinds of 4-mer subsequences in the RNA sequence. There are 7³=343 kinds of 3-mer subsequences and 7⁴=2401 kinds of 4-mer subsequences in the protein sequence. For example, AAA and AUC are two 3-mer subsequences of the RNA sequence, and AAAA and AAAU are two 4-mer subsequences of the RNA sequence. 111 and 112 are two 3-mer subsequences of the protein sequence, and 1111 and 1122 are the two 4-mer subsequences of the protein sequence. In other examples, the value of k may be 3 only or 4 only, which is not specifically limited in the present disclosure.

In step S420, an average value of the number of occurrences of each k-mer subsequence in each RNA-protein pair is calculated, and a variance of each k-mer subsequence is calculated according to the average value of the number of occurrences.

In an example embodiment, all 3-mer subsequences and 4-mer subsequences of the RNA sequence and the protein sequence can be obtained according to step S410, that is, 64 kinds of 3-mer subsequences and 256 kinds of 4-mer subsequences of the RNA sequence, and 343 kinds of 3-mer subsequences and 2401 kinds of 4-mer subsequences of the protein sequence are included. The average value of the number of occurrences of each 3-mer subsequence or 4-mer subsequence in each RNA-protein pair in the original data set may be calculated. The variance of each 3-mer subsequence or 4-mer subsequence may be calculated according to the average value of the number of occurrences. Before calculating the average value of the number of occurrences of each 3-mer subsequence or 4-mer subsequence in each RNA-protein pair in the original data set, the RNA sequence and the protein sequence of each RNA-protein pair in the original data set needs to be converted into 3-mer subsequences and 4-mer subsequences. For example, for an RNA sequence “AGAUGG”, the 3-mer subsequences of the sequence may include: “AGA”, “GAU”, “AUG” and “UGG”, and the 4-mer subsequence of the sequence may include: “AGAU”, “GAUG” and “AUGG”. That is, the corresponding 3-mer subsequences or 4-mer subsequences may be obtained by reading the RNA sequence in a forward overlap. Similarly, the corresponding 3-mer subsequences or 4-mer subsequences may also be obtained by reading the RNA sequence by reverse overlap. For example, the 3-mer subsequence of the sequence may include: “GGU”, “GUA”, “UAG” and “AGA”, and the 4-mer subsequences of the sequence may include: “GGUA”, “GUAG” and “UAGA”. In some embodiments, the RNA sequence may also be read in a non-overlapping manner to obtain the corresponding 3-mer subsequences or 4-mer subsequences. For example, the 3-mer subsequences of the sequence may include: “AGA” and “UGG”, which is not specifically limited in the present disclosure.

Exemplarily, by traversing the original data set, the number of occurrences of each 3-mer subsequence and/or 4-mer subsequence in each RNA-protein pair may be determined. The total number of occurrences of the subsequence in the original data set may be obtained by counting the number of occurrences of each 3-mer subsequence and/or 4-mer subsequence in each RNA-protein pair. The average value of the frequency of occurrences of each 3-mer subsequence and/or 4-mer subsequence in each RNA-protein pair may be obtained according to the total number of occurrences. Finally, the variance of each 3-mer subsequence and/or 4-mer subsequence may be calculated according to the average value of the number of occurrences of each 3-mer subsequence and/or 4-mer subsequence in each RNA-protein pair and the number of occurrences of each 3-mer subsequence and/or 4-mer subsequence in each RNA-protein pair.

For example, for the i-th k-mer subsequence, the subsequence may be a 3-mer subsequence of an RNA sequence or a protein sequence, or a 4-mer subsequence of an RNA sequence or a protein sequence. The total number of occurrences of this subsequence in the RPI1807 data set may be counted first. For example, the n RNA-protein pairs (n=3243) in the RPI1807 data set may be cycled, and the number of occurrences of the subsequence in each RNA-protein pair may be counted as: x₁, x₂, . . . , xₙ, and x₁, x₂, . . . , xₙ are summed to obtain the total number of occurrences of the subsequence in the RPI1807 data set, denoted as numᵢ. Then, the average value mᵢ of number of occurrences of the subsequence in each RNA-protein pair can be calculated according to the total number of occurrences numᵢ, that is, the average value of number of occurrences of the i-th k-mer subsequence in each RNA-protein pair is calculated according to the following formula:

$${m}_{i} = \frac{{num}_{i}}{n} \qquad ( 1 )$$

The variance of the i-th k-mer subsequence may be calculated from the average value of the number of occurrences of the i-th k-mer subsequence in each RNA-protein pair and the number of occurrences in each RNA-protein pair, that is, the variance s² of the i-th k-mer subsequence is calculated according to:

$${s}^{2} = \frac{{( {m}_{i} - {x}_{1} )}^{2} + {( {m}_{i} - {x}_{2} )}^{2} + … + {( {m}_{i} - {x}_{n} )}^{2}}{n} \qquad ( 2 )$$

where n is the number of RNA-protein pairs in the RPI1807 data set, mᵢ is the average value of the number of occurrences of the subsequence in each RNA-protein pair, and xₙ is the number of occurrences of the subsequence in the n-th RNA-protein pair, similarly, x₁ is the number of occurrences of the subsequence in the first RNA-protein pair, and x₂ is the number of occurrences of the subsequence in the second RNA-protein pair.

In step S430, the original sequence feature set is determined according to the variance of each k-mer subsequence.

After calculating the variance of each k-mer subsequence, k-mer subsequences that meet a preset condition may be determined according to the variance of each k-mer subsequence, and the original sequence feature set is composed of the k-mer subsequences that meet the preset condition. Exemplarily, all 3-mer subsequences and 4-mer subsequences of the RNA sequences, and all 3-mer subsequences and 4-mer subsequences of the protein sequences may be ranked according to their variances, such as in a descending or. Top-ranked k-mer subsequences may be selected to constitute the original sequence feature set. For example, the top 560 k-mer subsequences may be selected, and the original sequence feature set may be constituted by these 560 k-mer subsequences. The 560 k-mer subsequences may include the 3-mer subsequences of the top 60 RNA sequences, the 4-mer subsequences of the top 200 RNA sequences, the 3-mer subsequences of the top 200 protein sequences, and the 4-mer subsequences of the top 100 protein sequences. It can be understood that the number of selected k-mer subsequences is only illustrative, and any number of k-mer subsequences can be selected according to actual requirements. In other examples, a variance threshold can be preset, and k-mer subsequences with variance greater than the threshold may be selected, and the selected k-mer subsequences form the original sequence feature set. For example, when the preset variance threshold is 3, k-mer subsequences with variance greater than 3 can be selected to form the original sequence feature set. It should be noted that when performing feature selection, a feature with a large variance can be selected, and a large variance indicates that the data of the feature has a large difference, that is, the sample can be better distinguished by using this feature, which can further improve the classification and prediction ability of the interaction prediction model.

In another example embodiment, the relative frequency of occurrence of each k-mer subsequence in the original data set may be counted, and the variance of each k-mer subsequence may be calculated according to the relative frequency of occurrence, and then the original sequence feature set may be determined according to the magnitude of the variance of each k-mer subsequence.

Exemplarily, the frequency of occurrence of each k-mer subsequence in the original data set may be counted, and the relative frequency of occurrence of each k-mer subsequence in the original data set may be calculated according to the frequency of occurrences. For example, the relative frequency of occurrence of a subsequence in the original data set may be obtained by calculating the ratio between the frequency of occurrences and the total number of RNA-protein pairs in the original data set. By traversing the original data set, each k-mer subsequence was marked for presence in each RNA-protein pair. The variance of each k-mer subsequence may be calculated from the relative frequency of occurrence of each k-mer subsequence in the original data set and the marker value of each k-mer subsequence in each RNA-protein pair.

For example, for the i-th k-mer subsequence, the frequency of occurrence of the subsequence in the RPI1807 data set may be counted first. For example, N RNA-protein pairs (N=3243) in the RPI1807 data set may be cycled, if the subsequence appears in a current RNA-protein pair, the frequency of occurrence is incremented by 1, and if it does not appear in the current RNA-protein pair, the frequency of occurrence remains unchanged. The frequency of occurrence of the i-th k-mer subsequence in the RPI1807 data set is denoted as numᵢ, and then the relative frequency of occurrences of the subsequence in the RPI1807 data set may be calculated according to the frequency of occurrence numᵢ, that is, the relative frequency of occurrence Freqᵢ in each RNA-protein pair, which is:

$${Freq}_{i} = \frac{{num}_{i}}{N} \qquad ( 3 )$$

After determining the relative frequency of occurrence of the i-th k-mer subsequence in the RPI1807 data set, the occurrence of this subsequence in each RNA-protein pair may be viewed by traversing the RPI1807 data set, and the occurrence may be marked, denoted as Appearᵢ_{ₙ}. That is, if the subsequence appears in the n-th RNA-protein pair, the marker value Appearᵢ_{ₙ}=1, and if it does not appear in the n-th RNA-protein pair, the marker value Appearᵢ_{ₙ}=0.

After obtaining the relative frequency of occurrence Freqᵢ of the subsequence in the RPI1807 data set and the marker value Appearᵢ_{ₙ} in the n-th RNA-protein pair, the square of the difference between the marker value of the i-th k-mer subsequence in each RNA-protein pair in the RPI1807 data set and the relative frequency of occurrence of the k-mer subsequence in the RPI1807 data set is summed to obtain the variance Varᵢ of the i-th k-mer subsequence in the RPI1807 data set according to:

$${Var}_{i} = {\sum }_{n = 1}^{n = N} {( {Appear}_{{i}_{n}} - {Freq}_{i} )}^{2} \qquad ( 4 )$$

where Appearᵢ_{ₙ} is the marker value of the i-th k-mer subsequence in the n-th RNA-protein pair, Freqᵢ is the relative frequency of occurrence of the i-th k-mer subsequence in the RPI1807 data set, and N is the total number of RNA-protein pairs in the RPI1807 data set.

After calculating the variance of each k-mer subsequence, exemplarily, all 3-mer subsequences and 4-mer subsequences of RNA sequences and all 3-mer subsequences and 4-mer subsequences of protein sequences can be ranked according to the variance, such as in a descending order, and the top-ranked k-mer subsequences may be selected to form the original sequence feature set. In some other examples, a variance threshold may be preset, and k-mer subsequences with variance greater than the threshold may be selected, and the selected k-mer subsequences form the original sequence feature set.

In an example embodiment of the present disclosure, the k-mer features of each RNA-protein pair in the original data set may be extracted, and the original sequence feature set is composed of the extracted k-mer features of the RNA sequences and the k-mer features of the protein sequences. Taking the k-mer features of an RNA sequence as an example, a k-mer feature may contain the monomer component information of the RNA sequence (that is, the individual bases contained) and the sequence order information. Therefore, the use of k-mer features may better describe an RNA sequence, that is, an RNA sequence may be more accurately determined according to the k-mer features, and different RNA sequences may also be distinguished by the k-mer features. In order to further mine the relationship between RNA sequences and protein sequences, the frequent itemset features of each RNA-protein pair in the original data set may also be extracted, and the original sequence feature set is composed of the extracted frequent itemset features. The frequent itemset feature may combine the kmer features of RNA sequences with the kmer features of protein sequences. Therefore, using frequent itemset features can better distinguish between interacting and non-interacting RNA-protein pairs. It is also possible to extract k-mer features and frequent itemset features at the same time, and combine them to form the original sequence feature set. By combining the characteristics of k-mer features and frequent itemset features, the interaction between RNA and protein in an unknown RNA-protein pair can be predicted more accurately, and embodiments of the present disclosure do not impose specific limitations on this.

The frequent itemset feature refers to a k-mer subsequence pair composed of an RNA k-mer subsequence and a protein k-mer subsequence with a certain support in the original data set. The support refers to a percentage of an item including both A and B in all items. For example, a subsequence pair (AAU, 137) represents a 3-mer subsequence pair consisting of a 3-mer subsequence AAU of an RNA and a 3-mer subsequence 137 of a protein. The support of this subsequence pair is the percentage of RNA-protein pairs that contain both subsequences AAU and 137 to all RNA-protein pairs in the original data set.

In an example embodiment, the RNA sequence and protein sequence in each RNA-protein pair can be converted to k-mer subsequences, respectively, to obtain k-mer subsequence pairs. The relative frequency of occurrence of each k-mer subsequence pair in the original data set is counted, and the k-mer subsequence pairs that meet a preset condition for relative frequency of occurrence are used as frequent itemset features to form the original sequence feature set.

For example, the RNA sequences and protein sequences of all positive RNA-protein pairs in the RPI1807 data set may be converted into positive 3-mer subsequences and positive 4-mer subsequences, respectively. Similarly, the RNA sequences and protein sequences of all negative RNA-protein pairs in this data set may be converted into negative 3-mer subsequences and negative 4-mer subsequences, respectively. By traversing the RPI1807 data set, all positive and negative RNA 3-mer subsequences, positive and negative RNA 4-mer subsequences, positive and negative protein 3-mer subsequences, and positive and negative protein 4-mer subsequences in the data set may be found. The RNA 3-mer subsequences and protein 3-mer subsequences, RNA 4-mer subsequences and protein 4-mer subsequences in the data set are cross-combined in pairs to obtain a variety of 3-mer subsequence pairs and 4-mer subsequence pairs. Exemplarily, a positive RNA 3-mer subsequence and a positive protein 3-mer subsequence may be cross-combined to obtain a positive 3-mer subsequence pair. A negative RNA 3-mer subsequence and a negative protein 3-mer subsequence may be cross-combined to obtain a negative 3-mer subsequence pair. A positive RNA 4-mer subsequence and a positive protein 4-mer subsequence may be cross-combined to obtain a positive 4-mer subsequence pair. A negative RNA 4-mer subsequence and a negative protein 4-mer subsequence may be cross-combined to obtain a negative 4-mer subsequence pair.

The relative frequency of occurrence of each subsequence pair in the data set may be counted. For example, for any positive 3-mer subsequence pair, the relative frequency of occurrence Freq of the positive 3-mer subsequence pair in the data set is calculated according to:

$$Freq = \frac{num}{NUM} \qquad ( 5 )$$

where num is the number of occurrences of the positive 3-mer subsequence pair in the data set, and NUM is the total number of occurrences of all positive 3-mer subsequence pairs in the data set.

After calculating the relative frequency of occurrence of each k-mer subsequence pair in the original data set, exemplarily, all 3-mer subsequence pairs and 4-mer subsequence pairs may be ranked according to the magnitude of the relative frequency of occurrence, such as in a descending order, and top-ranked k-mer subsequence pairs may be selected to form a frequent itemset. For example, all positive 3-mer subsequence pairs are ranked in a descending order, the first m 3-mer subsequence pairs may be selected to form a frequent itemset A1. All positive 4-mer subsequence pairs may be ranked in a descending order, and first n 4-mer subsequence pairs may be selected to form a frequent itemset A2. All negative 3-mer subsequence pairs may be ranked in a descending order, and first p 3-mer subsequence pairs may be selected to form a frequent itemset A3. All negative 4-mer subsequence pairs may be ranked in a descending order, and the first q 4-mer subsequence pairs may be selected to form a frequent itemset A4. Then, the original sequence feature set may be formed by the four frequent itemsets A1, A2, A3 and A4. In other examples, a relative frequency of occurrence threshold may be preset, and k-mer subsequence pairs whose relative frequency of occurrence is greater than the threshold may be selected, and the selected k-mer subsequence pairs may be used as frequent itemset features to form the original sequence feature set, and the present disclosure does not specifically limit this.

In another example embodiment, the RNA sequence and protein sequence in each RNA-protein pair may be converted into k-mer subsequences, respectively, and a first candidate itemset may be composed of the k-mer subsequences, and the k-mer subsequences include RNA k-mer subsequences and protein k-mer subsequences. For example, the RNA sequence and protein sequence of each RNA-protein pair in the RPI1807 data set may be first converted into 3-mer subsequences and 4-mer subsequences, respectively. By traversing the RPI1807 data set, all RNA 3-mer subsequences, RNA 4-mer subsequences, protein 3-mer subsequences and protein 4-mer subsequences in this data set may be found, and all 3-mer subsequences and 4-mer subsequences in this data set form a first candidate itemset C1.

The relative frequency of occurrence of each k-mer subsequence in the first candidate itemset C1 in the original data set may be counted. Exemplarily, for the j-th k-mer subsequence, the subsequence may be a 3-mer subsequence of an RNA sequence or a protein sequence, or a 4-mer subsequence of an RNA sequence or a protein sequence. The number of occurrences of this subsequence in the RPI1807 data set may be counted first. For example, N RNA-protein pairs in the RPI1807 data set may be cycled, if the subsequence appears in the current RNA-protein pair, the number of occurrences is incremented by 1, and if it does not appear in the current RNA-protein pair, the number of occurrences remains unchanged. The number of occurrences of the j-th k-mer subsequence in the RPI1807 data set is denoted as numⱼ, and then the relative frequency of occurrence Freqⱼ of the subsequence in the RPI1807 data set may be calculated according to the number of occurrences numⱼ, that is:

$${Freq}_{j} = \frac{{num}_{j}}{N} \qquad ( 6 )$$

Similarly, the relative frequency of occurrence of each 3-mer subsequence or 4-mer subsequence in the first candidate itemset C1 in the RPI1807 data set may be calculated. Then, all 3-mer subsequences and 4-mer subsequences may be screened according to a preset threshold for relative frequency of occurrence. For example, RNA 3-mer subsequences with a relative frequency of occurrence greater than a first threshold, RNA 4-mer subsequences with a relative frequency of occurrence greater than a second threshold, protein 3-mer subsequences with a relative frequency of occurrence greater than a third threshold, and protein 4-mer subsequences with a relative frequency of occurrence greater than a fourth threshold may together form a frequent itemset L1. The first threshold, the second threshold, the third threshold and the fourth threshold may be the same or different, which are not specifically limited in the present disclosure. In other examples, the relative frequencies of occurrence of the 3-mer subsequences and the 4-mer subsequences of the RNA sequence and the relative frequencies of occurrence of the 3-mer subsequences and the 4-mer subsequences of the protein sequence may be ranked in a descending order, and to-ranked subsequences may form the frequent itemset L1, which is not specifically limited in the present disclosure.

The RNA 3-mer subsequences and protein 3-mer subsequences, RNA 4-mer subsequences and protein 4-mer subsequences in the frequent itemset L1 may be cross-combined in pairs to obtain a variety of 3-mer subsequence pairs and 4-mer subsequence pairs, and a second candidate itemset C2 may be composed of the multiple subsequence pairs obtained by combination. For example, if a frequent itemset includes [AAU, AUC, 137, 123, AAUU, AGUC, 1737, 1234], “AAU” and “137” may be combined to obtain a 3-mer subsequence pair “AAU_137”, or “AAU” and “123” may be combined to obtain a 3-mer subsequence pair “AAU_123”. Similarly, by cross-combining the RNA 3-mer subsequences and the protein 3-mer subsequences, a 3-mer subsequence pairs “AUC_137” and “AUC_123” may also be obtained. By cross-combing the RNA 4-mer subsequences and the protein 4-mer subsequences, 4-mer subsequence pairs “AAUU_1737”, “AAUU_1234”, “AGUC_1737” and “AGUC_1234” may be obtained.

The relative frequency of occurrence of each subsequence pair in the second candidate itemset C2 in the RPI1807 data set may be counted. Exemplarily, for the f-th k-mer subsequence pair, the subsequence pair may be a 3-mer subsequence pair or a 4-mer subsequence pair. The number of occurrences of this subsequence pair in the RPI1807 data set may be counted first. For example, N RNA-protein pairs in the RPI1807 data set may be cycled, and if the subsequence pair appears in the current RNA-protein pair, the number of occurrences is incremented by 1, and if it does not appear in the current RNA-protein pair, the number of occurrences remains unchanged. The frequency of occurrence of the f-th k-mer subsequence pair in the RPI1807 data set is denoted as num_{f}, and then the relative frequency of occurrence of the subsequence pair in the RPI1807 data set may be calculated according to the frequency of occurrence num_{f}, That is, the support support_{f} of the subsequence pair is obtained, which is:

$${support}_{f} = \frac{{num}_{f}}{N} \qquad ( 7 )$$

Similarly, the support of each subsequence pair in the second candidate itemset C2 may be calculated.

After the support of each subsequence pair is calculated, the subsequence pairs satisfying a preset condition can be determined according to the support of each subsequence pair, and the original sequence feature set is composed of the subsequence pairs satisfying the preset condition. Exemplarily, a support threshold may be preset, and subsequence pairs with a support greater than the threshold are selected, and the selected subsequence pairs form the original sequence feature set. For example, there are 370 sub-sequence pairs with a support greater than the threshold, these 370 sub-sequence pairs are 370 frequent itemset features, and the original sequence feature set may be composed of the 370 frequent itemset features. In other examples, all subsequence pairs in the second candidate itemset C2 may be ranked in a descending order according to theirs supports, and the top-ranked subsequence pairs are selected to form the original sequence feature set, which is not specifically limited in this disclosure.

In an example embodiment of the present disclosure, the frequent itemset features can combine the kmer features of RNA sequence and the kmer features of protein sequence together, and the frequent itemset features can better distinguish RNA-protein pairs with and without interaction. Therefore, when the original sequence feature set is composed of frequent itemset features and the feature extraction of the RNA-protein pair to be predicted is performed based on the original sequence feature set, whether the RNA-protein pair to be predicted has an interaction can be more accurately determined based on the extracted sequence features of the RNA-protein pair to be predicted.

In step S320, the sequence features of the RNA-protein pair to be predicted are determined according to the original sequence feature set.

In an example embodiment, the RNA sequence and protein sequence in the RNA-protein pair to be predicted may be converted into k-mer subsequences respectively, and after obtaining the original sequence feature set, each k-mer subsequence may be searched in the original sequence feature set, and the sequence features of the RNA-protein pair to be predicted may be obtained according to the search result. The sequence features of the RNA-protein pair to be predicted may refer to complete sequence features consisting of RNA sequence features and protein sequence features.

Exemplarily, the original sequence feature set may consist of 560 k-mer subsequences. For example, the 560 k-mer subsequences may be [CCC, . . . , AGU, CCCC, . . . , CUGG, 777, . . . , 373, 7774, . . . , 7571]. The RNA-protein pair to be predicted is converted into 3-mer subsequences and 4-mer subsequences to obtain RNA 3-mer subsequences, RNA 4-mer subsequences, protein 3-mer subsequences and protein 4-mer subsequences. Feature calculation may be performed on the RNA sequence and protein sequence in the RNA-protein pair to be predicted based on the original sequence feature set to obtain the sequence features of the RNA-protein pair to be predicted.

Specifically, for the original sequence feature set [CCC, . . . , AGU, CCCC, . . . , CUGG, 777, . . . , 373, 7774, . . . , 7571] including 560 feature dimensions, the feature of each feature dimension corresponds a k-mer subsequence. For example, the subsequence CCC is the feature of the first feature dimension, and the subsequence 7571 is the feature of the 560-th feature dimension. All 3-mer subsequences and 4-mer subsequences of the RNA-protein pair to be predicted may be searched in the original sequence feature set, and whether there exists a feature in each feature dimension in the original sequence feature set is determined according to the search result. If a feature in a feature dimension exists, the feature value for the feature dimension is 1, and if it does not exist, the feature value for the feature dimension is 0. For example, if the RNA sequence in the RNA-protein pair to be predicted is “CCACCCCAAUA” and the protein sequence is “123373777373”, the corresponding RNA 3-mer subsequences include CCA, . . . , CCC, . . . , CCA, . . . , AUA, and RNA 4-mer subsequences include CCAC, . . . , CCCC, . . . , AAUA, and protein 3-mer subsequences include 123, . . . , 373, . . . , 777, . . . , 373, and protein 4-mer subsequences include 1233, . . . , 7377, . . . , 7373. The search result of each subsequence in the original sequence feature set may be as shown in Table 1:

| | RNA 3-mer | | | RNA 4-mer | | | protein 3-mer | | | protein 4-mer | | |
|---|---|---|---|---|---|---|---|---|---|---|---|---|
| | subsequences | | | subsequences | | | subsequences | | | subsequences | | |
| number | (top-60) | | | (top-200) | | | (top-200) | | | (top-200) | | |
| features | CCC | . . . | AUA | CCCC | . . . | CUGG | 777 | . . . | 373 | 7774 | . . . | 7571 |
| feature | 1 | . . . | 1 | 1 | . . . | 0 | 1 | . . . | 1 | 0 | . . . | 0 |
| values | | | | | | | | | | | | |

Table 1

It can be seen from Table 1 that the feature CCC of the first feature dimension in the original sequence feature set is also a 3-mer subsequence of the RNA-protein pair. Therefore, it can be determined that the feature CCC in the first feature dimension in the original sequence feature set exists, and the corresponding feature value may be denoted as 1. For another example, if the feature CUGG in the original sequence feature set does not exist in the RNA sequence of the RNA-protein pair to be predicted, the feature value in the 260-th feature dimension in the original sequence feature set may be recorded as 0. Finally, a 560-dimensional feature value vector [1, . . . , 1, 1, . . . , 0, 1, . . . , 1, 0, . . . , 0] can be calculated, which is the features of the RNA-protein pair to be predicted. It can be understood that each feature value contained in the feature value vector has a one-to-one correspondence with the feature value of each feature dimension in the original sequence feature set.

In this example embodiment, by extracting the k-mer features of each RNA-protein pair in the original data set, the original sequence feature set is composed of the extracted k-mer features of the RNA sequences and the k-mer features of the protein sequences. By performing feature extraction on the RNA-protein pair to be predicted based on the original sequence feature set, the sequence features of the RNA-protein pair to be predicted may be obtained. Taking the k-mer features of an RNA sequence as an example, the k-mer feature may include the monomer composition information (i.e., the individual bases contained) and sequence order information. Therefore, the k-mer features can be used to better describe an RNA sequence, that is, an RNA sequence can be more accurately determined according to the k-mer features, and different RNA sequences can also be distinguished by the k-mer features.

In another example embodiment, after obtaining the original sequence feature set, the RNA sequence and the protein sequence in the RNA-protein pair to be predicted may be converted into k-mer subsequences, respectively, and the RNA k-mer subsequences and protein k-mer subsequences are cross-combined to obtain multiple RNA-protein k-mer subsequence pairs. Exemplarily, after obtaining the RNA 3-mer subsequences, the protein 3-mer subsequences, the RNA 4-mer subsequences and the protein 4-mer subsequences of the RNA-protein pair to be predicted, the RNA 3-mer subsequences and the protein 3-mer subsequences, and RNA 4-mer subsequences and protein 4-mer subsequences may be cross-combined in pairs to obtain a variety of 3-mer subsequence pairs and 4-mer subsequence pairs. Each RNA-protein k-mer subsequence pair can be searched in the original sequence feature set, and the sequence features of the RNA-protein pair can be obtained according to the search results.

Exemplarily, the original sequence feature set may consist of 370 frequent itemset features. For example, the 370 frequent itemset features can be [CCA_121, . . . , UCUG_1312, . . . , AAU_122, . . . , CUUU_1312, . . . ]. The RNA-protein pair to be predicted is converted into 3-mer subsequences and 4-mer subsequences to obtain RNA 3-mer subsequences, RNA 4-mer subsequences, protein 3-mer subsequences and protein 4-mer subsequences. RNA 3-mer subsequences may be paired with protein 3-mer subsequences, RNA 4-mer subsequences may be paired with protein 4-mer subsequences, and various 3-mer subsequence pairs and 4-mer subsequence pairs may be obtained. Then, feature calculation may be performed on the RNA sequence and the protein sequence in the RNA-protein pair to be predicted based on the original sequence feature set to obtain the sequence features of the RNA-protein pair.

Specifically, for the original sequence feature set [CCA_121, . . . , UCUG_1312, . . . , AAU_122, . . . , CUUU_1312, . . . ] including 370 feature dimensions, the feature of each feature dimension corresponds a k-mer subsequence pair. For example, the subsequence pair CUG_122 is the feature of the first feature dimension. All subsequence pairs of the RNA-protein pair can be searched in the original sequence feature set, and whether the feature in each feature dimension in the original sequence feature set exists or not is determined according to the search result. If the feature in a feature dimension in the original sequence feature set exists, the feature value in the feature dimension is 1, and if it does not exist, the feature value in the feature dimension is 0. For example, if the RNA sequence in the RNA-protein pair to be predicted is “CCAUCUGAAU”, the protein sequence is “1312137122”. It can be seen that CCA_121, UCUG_1312, and AAU_122 in the subsequence pair of the RNA-protein pair exist in the original sequence feature set. Therefore, the feature value of the corresponding feature dimension in the original sequence feature can be recorded as 1. The search results of each sub-sequence pair in the original sequence feature set can be as shown in Table 2:

| | positive 3-mer | | positive 4-mer | | negative 3-mer | | negative 3-mer | |
|---|---|---|---|---|---|---|---|---|
| | subsequence pair | | subsequence pair | | subsequence pair | | subsequence pair | |
| number | (top-40) | | (top-130) | | (top-100) | | (top-100) | |
| features | CCA_121 | . . . | UCUG_1312 | . . . | AAU_122 | . . . | CUUU_1312 | . . . |
| feature | 1 | . . . | 1 | . . . | 1 | . . . | 0 | . . . |
| values | | | | | | | | |

Table 2

Finally, a 370-dimensional feature value vector [1, . . . , 1, . . . , 1, . . . , 0, . . . ] can be calculated, and the feature value vector is the sequence features of the RNA-protein pair to be predicted. Similarly, each feature value contained in the feature value vector has a one-to-one correspondence with the feature of each feature dimension in the original sequence feature set.

In another example embodiment, after obtaining the original sequence feature set, the RNA sequence and the protein sequence in the RNA-protein pair to be predicted may be converted into k-mer subsequences respectively, and each k-mer subsequence may be searched in the original sequence feature set to obtain a first sequence feature. Then, the RNA k-mer subsequences and the protein k-mer subsequences may be combined to obtain a variety of RNA-protein k-mer subsequence pairs, and each RNA-protein k-mer subsequence pair is searched in the original sequence feature set to obtain a second sequence feature. Finally, the sequence feature of the RNA-protein pair to be predicted may be composed of the first sequence feature and the second sequence feature.

Exemplarily, the original sequence feature set may include two feature subsets, the two feature subsets respectively include 560 kinds k-mer subsequences [CCC, . . . , CCCC, . . . , 777, . . . , 7774, . . . ] and 370 frequent itemset features [CCA_121, . . . , UCUG_1312, . . . , AAU_122, . . . , CUUU_1312, . . . ]. The RNA-protein pair to be predicted may be converted into RNA 3-mer subsequences, RNA 4-mer subsequences, protein 3-mer subsequences and protein 4-mer subsequences, and at the same time, RNA 3-mer subsequences may be paired with protein 3-mer subsequences, and RNA 4-mer subsequences may be paired with protein 4-mer subsequences, so as to obtain a variety of 3-mer subsequence pairs and 4-mer subsequence pairs. Then, feature calculation may be performed on the RNA sequence and protein sequence in the RNA-protein pair to be predicted based on the original sequence feature set, to obtain the sequence features of the RNA-protein pair to be predicted.

Specifically, all subsequences and subsequence pairs of the RNA-protein pair to be predicted may be searched in the original sequence feature set, and whether a feature in each feature dimension of the original sequence feature set exists is determined according to the search result. For example, by searching all subsequences of the RNA-protein pair to be predicted, a 560-dimensional feature value vector [1, . . . , 1, . . . , 1, . . . , 0, . . . ] may be calculated, i.e., the first sequence feature. By searching all subsequence pairs of the RNA-protein pair to be predicted, a 370-dimensional feature value vector [1, . . . , 1, . . . , 1, . . . , 0, . . . ] may be calculated, i.e., the second sequence feature. For example, by concatenating the two feature value vectors, a 930-dimensional feature value vector can be obtained, which is the sequence features of the RNA-protein pair to be predicted. The two feature value vectors may also be directly input into the interaction prediction model at the same time, which is not specifically limited in the present disclosure.

In other examples, a 930-dimensional original sequence feature set may be composed of 560 k-mer subsequences and 370 frequent itemset features. For example, the original sequence feature set is [CCC, . . . , CCCC, . . . , 777, . . . , 7774, . . . , CCA_121, . . . , UCUG_1312, . . . , AAU_122, . . . , CUUU_1312, . . . ]. All subsequences and subsequence pairs of the RNA-protein pair may be searched in the original sequence feature set, and according to the search results, whether the feature in each feature dimension of the original sequence feature set exists may be determined, and a 930-dimensional feature value vector [1, . . . , 1, . . . , 1, . . . , 0, . . . , 1, . . . , 1, . . . , 1, . . . , 0, . . . ] may be calculated, which is the sequence features of the RNA-protein pair to be predicted.

In step S230, the RNA-protein pair to be predicted is vectorized to obtain the RNA sequence representation vector and the protein sequence representation vector in the RNA-protein pair to be predicted.

After feature extraction is performed on the RNA-protein pair to be predicted, the obtained sequence features may be used as the input of a first interaction prediction model. In order to obtain the sequence information of the RNA sequence and the protein sequence and the information between adjacent bases and amino acids, the RNA-protein pair to be predicted may be vectorized, and the obtained vector may be used as the input of a second interaction prediction model.

In an example embodiment, the RNA sequence and the protein sequence in the RNA-protein pair to be predicted may be converted to k-mer subsequences, respectively. For example, the RNA sequence may be divided into M RNA k-mer subsequences in a non-overlapping manner, and the protein sequence may be divided into N protein k-mer subsequences in a non-overlapping manner. For example, if the RNA sequence is AUCUGAAAU, it can be divided into three RNA k-mer subsequences, namely AUC, UGA and AAU. It can be understood that the non-overlapping division of the RNA sequence and the protein sequence into multiple k-mer subsequences is to vectorize the RNA sequence and protein sequence, that is, the bases in the RNA sequence and the amino acids in the protein sequence are vectorized in the form of k-mers. Similarly, in other examples, each base included in the RNA sequence in the RNA-protein pair to be predicted may be vectorized to obtain multiple base vectors, and the RNA sequence representation vector can be obtained by concatenating the multiple base vectors. Also, each amino acid vector included in the protein sequence in the RNA-protein pair to be predicted may be vectorized to obtain multiple amino acid vectors, and the protein sequence representation vector can be obtained by concatenating the multiple amino acid vectors. The RNA sequence may alternatively be divided into P RNA k-mer subsequences in an overlapping manner, and the protein sequence may be divided into Q protein k-mer subsequences in the overlapping manner, which are not specifically limited in the present disclosure. The RNA sequence representation vector and the protein sequence representation vector may then be input into the second interaction prediction model, respectively.

Specifically, each k-mer subsequence of the RNA sequence and the protein sequence may be encoded first. Exemplarily, when k=3, there may be 64 RNA 3-mer subsequences and 343 protein 3-mer subsequences. Each RNA 3-mer subsequence and protein 3-mer subsequence may be encoded by Embedding (vector mapping) in turn, and each 3-mer subsequence is represented by a low-dimensional vector, and corresponding multiple 3-mer subsequence vectors can be obtained. In one example, One-Hot encoding may be performed on each 3-mer subsequence. One-Hot encoding is also known as one-bit effective encoding. The method uses an N-bit state register to encode N states, each state has an independent register bit, and at any time, only one bit in the register is valid. For example, for the i-th RNA 3-mer subsequence, that is, the RNA 3-mer subsequence whose index is an integer i, a 64-dimensional One-Hot vector can be obtained by encoding, and the i-th element in the vector is set to 1, other elements are set to 0, in the form of [0, 1, 0, 0, . . . , 0]. For the j-th protein 3-mer subsequence, that is, the protein 3-mer subsequence whose index is integer j, a 343-dimensional One-Hot vector can be obtained by encoding, and the j-th element in the vector is set to 1, and all other elements are set to 0. Similarly, each RNA 3-mer subsequence and protein 3-mer subsequence may correspond to a 3-mer One-Hot vector. In other examples, a dense vector may be used to represent each 3-mer subsequence. For example, Word2vec algorithm may be used to map each 3-mer subsequence into a vector space, and each 3-mer subsequence may be represented by a subsequence vector in this vector space. Each 3-mer subsequence may be encoded with a BERT (Bidirectionally Encoded Representation from Transformer) pre-training model to obtain corresponding multiple 3-mer subsequence vectors. Specifically, large-scale RNA sequence data may be obtained, and the BERT pre-training model may be used for training. After the training is completed, a high-dimensional vector of the RNA sequence may be obtained by inputting a certain RNA sequence into the trained model, which is not specifically limited in the present disclosure.

After obtaining all 3-mer One-Hot vectors of the RNA sequence and the protein sequence, the RNA sequence and the protein sequence in the RNA-protein pair to be predicted may be converted into 3-mer subsequences, for example, to obtain M RNA 3-mer subsequences and N protein 3-mer subsequences. Then, the M 3-mer One-Hot vectors corresponding to the M RNA 3-mer subsequences may be determined by query, and the M 3-mer One-Hot vectors may be concatenated in turn, for example, the concatenating may be performed in the row direction, to obtain an M*64 two-dimensional matrix, such as:

$$[ [ 0 , 1 , 0 , 0 , … . , 0 ] ; [ 0 , 0 , 0 , 1 , … . , 0 ] ; … ; [ 1 , 0 , 0 , 0 , … . , 0 ] ]$$

The two-dimensional matrix is the 3-mer One-Hot representation vector of the RNA sequence. The N 3-mer One-Hot vectors corresponding to the N protein 3-mer subsequences may also be determined by query, and the N 3-mer One-Hot vectors are concatenated in turn in the row direction to obtain a N*343 two-dimensional matrix. The two-dimensional matrix is the 3-mer One-Hot representation vector of the protein sequence. It is understandable that M 3-mer One-Hot vectors or N 3-mer One-Hot vectors may also be column concatenated, and 3-mer One-Hot vectors of the sequence may also be obtained by direct (that is, tail) concatenating, which is not specifically limited in the present disclosure.

In the example embodiment of the present disclosure, by vectorizing the RNA sequence and the protein sequence in the RNA-protein pair to be predicted, the RNA sequence representation vector and the protein sequence representation vector may be used as the input of a deep learning model, so as to further discover feature combinations which occur for very small number of times or which are new in the data, thereby revealing interactions between latent features.

In another example embodiment, each base vector included in the RNA sequence in the RNA-protein pair may be vectorized to obtain multiple base vectors, and the RNA sequence representation vector may be obtained by concatenating the multiple base vectors. Also, each amino acid vector included in the protein sequence in the RNA-protein pair may be vectorized to obtain multiple amino acid vectors, and the protein sequence representation vector may be obtained by concatenating multiple amino acid vectors.

Specifically, Embedding encoding may be first performed on the 4 types of bases (A, C, G and U) that may be included in the RNA sequence and the 7 types of amino acids (1, 2, 3, 4, 5, 6 and 7) that may be included in the protein sequence, and each base and each type of amino acid are represented by a low-dimensional vector, and corresponding multiple vectors may be obtained. In one example, One-Hot encoding may be performed on each base and each type of amino acid. For example, base A may be represented by a One-Hot vector [1, 0, 0, 0], and U may be represented by [0, 0, 0, 1]. Type 1 protein may be encoded as [1, 0, 0, 0, 0, 0, 0] and type 7 protein may be encoded as [0, 0, 0, 0, 0, 0, 1]. In other examples, a dense vector may also be used to represent each 3-mer subsequence. For example, the Word2vec algorithm may be used to map each base and each type of amino acid into a vector space to obtain a corresponding vector representation. Similarly, the Doc2vec algorithm, the Glove algorithm, or the like may also be used to convert each base and each type of amino acid into a vector, which is not specifically limited in the present disclosure.

For the RNA-protein pair to be predicted, the RNA sequence in the RNA-protein pair may be represented by the One-Hot vector of a single base to obtain an L*4 matrix, where L is the sequence length of the RNA sequence, that is, the number of bases contained in the RNA sequence. Exemplarily, if the RNA sequence is “ACUGAUGC”, the One-Hot vectors of 8 bases may be obtained. Referring to Table 3, each column represents the One-Hot vector representation of a base. For example, these 8 One-Hot vectors may be concatenated in the row direction to obtain an 8*4 matrix, which is the One-Hot vector representation of the RNA sequence.

In step S240, based on the sequence features of the RNA-protein pair to be predicted, the RNA sequence representation vector and the protein sequence representation vector in the RNA-protein pair to be predicted, an interaction prediction model is used to obtain a predicted interaction value of the RNA-protein pair to be predicted.

In one example embodiment, the interaction of the RNA-protein pair to be predicted may be predicted using at least three interaction prediction models. Exemplarily, the interaction prediction models may be all traditional machine learning models, or may be all deep learning models, or the at least three interaction prediction models include both traditional machine learning models and deep learning models. The traditional machine learning model refer to processing natural data in raw form. For example, constructing a pattern recognition or machine learning system requires the use of specialized knowledge to extract features from raw data (such as pixel values of images) and convert them into an appropriate feature representation. Exemplarily, traditional machine learning models may include a linear regression model, a logistic regression model, a support vector machine model, a decision tree model, a K-Nearest Neighbor (KNN) model, a random forest model, and naive Bayesian model, etc. The deep learning model has the ability to automatically extract features, and may be composed of multiple processing layers to form a complex computing model, so as to automatically obtain data representation and multiple abstraction levels, which is a learning for feature representation. Exemplarily, the deep learning model may include a convolutional neural network model, a recurrent neural network model, or the like.

It should be noted that when the interaction prediction models are all traditional machine learning models, the RNA-protein pair to be predicted may not be vectorized, and only the sequence features obtained by feature extraction of the RNA-protein pair to be predicted may be used as inputs to various traditional machine learning models. When the interaction prediction models are all deep learning models, the feature extraction of the RNA-protein pair to be predicted may not be performed, but only the RNA sequence representation vector and the protein sequence representation vector obtained by vectorizing the RNA-protein pair to be predicted may be used as inputs to various deep learning models.

In an example embodiment, after obtaining the sequence features of the RNA-protein pair to be predicted and the RNA sequence representation vector and the protein sequence representation vector in the RNA-protein pair to be predicted, the sequence features of the RNA-protein pair to be predicted may be input into a first interaction prediction model to obtain a first predicted interaction value. At the same time, the RNA sequence representation vector and the protein sequence representation vector in the RNA-protein pair to be predicted may be input into a second interaction prediction model to obtain a second predicted interaction value. The used interaction prediction models may include at least one first interaction prediction model and at least two second interaction prediction models, or may include at least two first interaction prediction models and at least one second interaction prediction models.

In the example embodiment of the present disclosure, by training at least three interaction prediction models (i.e., weakly supervised models), multiple learning results are obtained, and the multiple learning results are fused to obtain a strongly supervised model. The fusion of various learning results can achieve better learning effect than a single model. Understandably, a strongly supervised model can be thought of as an overall model that actually contains multiple weakly supervised models. Therefore, it is possible to combine the first interaction prediction model and the second interaction prediction model, and perform fusion learning on the outputs of the multiple models. Also, each model is trained individually during model training. Exemplarily, the model parameters of each model can be optimized through a back-propagation algorithm, and a strongly supervised model can be obtained from each optimized model, which can solve the problem of low generalization ability and easy overfitting when using a single model to perform prediction.

In an example embodiment, the sequence features of the RNA-protein pair to be predicted may be input into a traditional machine learning model to obtain the first predicted interaction value. The RNA sequence representation vector and the protein sequence representation vector in the RNA-protein pair to be predicted may be input into the deep learning model to obtain the second predicted interaction value. It should be noted that each deep learning model may include at least two sub-deep learning models, which are respectively used for processing the RNA sequence and the protein sequence in RNA-protein pair. For example, the representation vector of the RNA sequence may be input into a first sub-deep learning model to obtain a first sequence feature. At the same time, the representation vector of the protein sequence may be input into a second sub-deep learning model to obtain a second sequence feature. Finally, the first sequence feature and the second sequence feature may be fused through the fully connected layer of each deep learning model to obtain the second predicted interaction value according to the fused feature. The used interaction prediction models may include at least one traditional machine learning model and at least two deep learning models, or may include at least two traditional machine learning models and at least one deep learning model.

For example, the traditional machine learning model may include at least one of an SVM model, an LR (Logistic Regression) model, and a random forest model, and the deep learning model may include at least one of a CNN model and a recurrent neural network model. The recurrent neural network model may be an LSTM model or a BILSTM (Bi-directional LSTM, bidirectional long short-term memory network) model.

The SVM model, the CNN model, and the BiLSTM model may be used as examples to predict the interaction of RNA-protein pair. Exemplarily, a 930-dimensional original sequence feature set may be composed of 560 k-mer subsequences and 370 frequent itemset features. Feature calculation is performed on the original sequence feature set according to the k-mer subsequences of the RNA-protein pair to be predicted to obtain a 930-dimensional feature value vector, which is used as the input of the SVM model to get a predicted interaction value y₁. At the same time, the RNA-protein pair to be predicted may also be vectorized to obtain the k-mer One-Hot vectors of the RNA sequence and the protein sequence respectively, and the k-mer One-Hot vectors are used as the input of the CNN model and the BiLSTM model to obtain predicted interaction values y₂ and y₃.

In the example embodiment of the present disclosure, first, for the SVM model, a feature extraction method and model parameters may be manually designed to make the model more interpretable. The CNN model and the BiLSTM model have good generalization ability, and can discover feature combinations in the data which occur for very few times or which are new, thereby revealing the interaction between latent features. Secondly, the CNN model can better capture the features but ignore the position information of the features, that is, the relationship between bases and amino acids at a certain interval cannot be extracted. The BiLSTM model has better memory ability, and can use the sequence information and position information of the data to make up for the shortcomings of the CNN model in memory ability. Finally, the SVM model is simple and has good interpretability. When using the CNN model, the BiLSTM model and the SVM model to predict the interaction of unknown RNA-protein pairs, the interpretability of the RPI prediction by the overall model can be enhanced. In general, the present disclosure can effectively combine the characteristics of each interaction prediction model, so that the prediction ability of the overall model can be improved.

In step S250, the interaction between the RNA and the protein is determined according to the predicted interaction values.

After obtaining at least three predicted interaction values, multiple predicted interaction values may be fused, and the interaction between RNA and protein may be determined according to the fusion result.

In an example embodiment, a predicted value threshold may be preset, according to which each predicted interaction value may be marked. When the predicted interaction value is greater than the predicted value threshold, it is marked as 1. When the predicted interaction value is less than the predicted value threshold, it is marked as 0. For example, the predicted value threshold is set to 0.6. If a predicted interaction value is greater than or equal to 0.6, the prediction result of the corresponding interaction prediction model is marked as 1, that is, the interaction prediction model predicts that the input RNA-protein pair has interaction; otherwise, the prediction result of the corresponding interaction prediction model is marked as 0. After marking the prediction results of the at least three interaction models, the individual marker values may be summed, i.e., the final predicted interaction value T is calculated according to the following formula:

$$T = {\sum }_{i = 1}^{i = n} {t}_{i}$$

where tᵢ is the marker value of the prediction result of the i-th interaction prediction model, and n is the number of the interaction prediction models. When n is an odd number, if T≥n/2, it indicates that the RNA-protein pair to be predicted has an interaction. If T<n/2, it indicates that the RNA-protein pair has no interaction. When n is an even number, if T≥n/2, it indicates that the RNA-protein pair to be predicted has interaction, and if T<n/2, it indicates that the RNA-protein pair to be predicted has no interaction. In other examples, when n is an even number, it can also be set as: T>n/2 indicating that the RNA-protein pair to be predicted has interaction, and T≤n/2 indicating that the RNA-protein pair to be predicted has no interaction with each other, which is not specifically limited in the present disclosure.

In another example implementation, weight parameters of multiple interaction prediction models may be obtained, and a fusion calculation may be performed on multiple predicted interaction values and corresponding weight parameters. For example, a weighted sum calculation may be performed on multiple predicted interaction values, and the interaction of the RNA-protein pair may be determined based on the computational result. For example, when using the SVM model, the CNN model and the BiLSTM model to predict the interaction of RNA-protein pair, the predicted interaction value yₒᵤₜ of the RNA-protein pair may be calculated according to:

$${y}_{out} = α * {y}_{1} + β * {y}_{2} + γ * {y}_{3}$$

where y₁ is the output value of the SVM model, y₂ is the output value of the CNN model, y₃ is the output value of the BiLSTM model, α, β, and γ are the weight parameters of the SVM model, the CNN model and the BILSTM model, respectively, and yₒᵤₜ may be any number between 0 and 1. Exemplarily, a boundary value of 0.5 can be used; when yₒᵤₜ>0.5, the prediction result may be marked as 1, which means that the RNA-protein pair has an interaction; when yₒᵤₜ≤0.5, the prediction result may be marked as 0, which means that the RNA-protein pair has no interaction. In the example embodiment of the present disclosure, the weight parameters may be set manually, for example, a larger weight may be set for an interaction prediction model with a higher accuracy rate, which is not specifically limited in this disclosure.

In the example embodiment of the present disclosure, referring to FIG. 5, the at least three interaction prediction models may be pre-trained according to steps S510 and S520 to realize the optimization of all model parameters in each prediction model. The final models obtained according to the training may make predictions for RNA-protein pairs whose interactions are unknown.

In step S510, a training data set is obtained. The training data set includes positive RNA-protein pairs and negative RNA-protein pairs.

All RNA-protein pairs in the original data set may be used as the training data set, or a part of the RNA-protein pairs in the original data set may be used as the training data set, or the original data set may be divided into a training data set, a validation data set, and a test data set in proportion. The training data set and the validation data set may be used to adjust the model parameters of each model, and multiple models with better performance may be obtained after training. The test data set can be used to test the generalization performance of each optimized model. Taking the RPI1807 data set as an example, there are a total of 3243 RNA-protein pairs in this data set, including 1807 positive pairs and 1436 negative pairs. Exemplarily, the data set may be divided into a training data set, a validation data set and a test data set according to a ratio of 7:2:1. The ratio of positive and negative cases in each data set may be consistent with the distribution of the overall data set, that is, the ratio is 1807:1436, which is about 1.25:1. For example, 1250 positive cases and 1000 negative cases may be selected as the training data set, 360 positive cases and 280 negative cases may be selected as the validation data set, and 180 positive cases and 140 negative cases may be selected as the test data set. It is understood that the number of RNA-protein pairs in the training data set is only indicative, and any number of RNA-protein pairs may be obtained to train each interaction prediction model multiple times to improve the performance of each interaction prediction model. It should be noted that the positive RNA-protein pairs may be marked, and the obtained marker value is “1”, which means that the RNA-protein pairs has interaction. The negative RNA-protein pairs may be marked, and the obtained marker value is “0”, which means that the RNA-protein pairs has no interaction.

In step S520, the training data set is used as the input of the interaction prediction models, and the model parameters of each interaction prediction model are iteratively updated. When the iteration termination condition is met, the training of all model parameters is completed, so as to use trained interaction prediction models to predict the interaction of the RNA-protein pair to be predicted.

Exemplarily, the training data set may be input into the at least three interaction prediction models, and the model parameters may be adjusted by using a back-propagation algorithm to obtain multiple weakly supervised models. For example, the model parameters may be weight parameters, bias parameters, penalty factors, etc. Exemplarily, the model parameters may be iteratively updated by using a stochastic gradient descent algorithm. According to the principle of back propagation, the objective function is continuously calculated, and the model parameters are updated according to the objective function. When the objective function converges to the minimum value, the training of the model parameters is completed. The model parameters may also be updated iteratively in the reverse direction, and when the preset number of iterations is satisfied, the training of all model parameters is completed. It should be noted that, the at least three interaction prediction models may be trained simultaneously, or the at least three interaction prediction models may be trained sequentially, which is not specifically limited in the present disclosure. However, each interaction prediction model is trained separately.

When optimizing the model parameters, a set of hyperparameters may be initialized first, and the at least three interaction prediction models may be continuously trained by using the training data set to obtain a first model. The hyperparameters may be the learning rate, the number of CNN layers, the size of the convolution kernel, etc. Then, the validation data set may be input into the trained first model to verify the prediction accuracy of the first model. When the prediction accuracy reaches a preset accuracy threshold, the current first model may be used as a second model, that is, the final training model is obtained. Finally, the test data set may be used on the trained model to test the final performance of the model. It can be understood that if the prediction effect of the second model is poor according to the test data set, a set of hyperparameters may be reset, and the interaction prediction model may be trained and verified in turn using the training data set and the verification data set again. When the prediction accuracy obtained by the trained interaction prediction model on the validation data set reaches the preset accuracy threshold, the final performance of the prediction model may be tested by using a new test data set.

After obtaining the weakly supervised model with better prediction accuracy, each RNA-protein pair in the test data set may be input into the weakly supervised model to judge the accuracy of the weakly supervised model. If the accuracy of the model is greater than the preset accuracy threshold, the training of the weakly supervised model is completed. The prediction results of multiple weakly supervised models may be fused to obtain a strongly supervised model, which may be used as the final prediction model to predict the interaction between unknown RNA-protein pairs. In other examples, the test data set may also be used to judge the Matthews correlation coefficient of the weakly supervised model. The Matthews correlation coefficient refers to the correlation coefficient between the actual classification and the predicted classification. Its value range is [0, 1]. The larger the value, the more related the predicted value and the actual value. The value of 1 indicates that the prediction result is absolutely correct. If the Matthews correlation coefficient of the model is greater than the preset threshold, it means that the training of the weakly supervised model is completed. The specificity rate, recall rate, and so on of the weakly supervised model may also be judged by using the test data set, which is not specifically limited in the present disclosure. It can be understood that if the accuracy of the weakly supervised model is not greater than the preset accuracy threshold, a new training data set may be obtained to train the model parameters of each interaction prediction model again, so as to continuously improve the model performance.

In the example embodiment of the present disclosure, a strongly supervised model is obtained by training multiple weakly supervised models, such as a traditional machine learning model, a convolutional neural network model, and a recurrent neural network model, and fusing the prediction results of the multiple weakly supervised models. The strong supervision model is simple and effective, and has low requirements on hardware equipment and a wide range of applications.

In an example implementation, the SVM model, the CNN model and the BiLSTM model may be trained separately.

Feature extraction is performed for each RNA-protein pair in the training data set, and the extracted sequence features may be sequentially input into the SVM model. Exemplarily, the original sequence feature set consists of 560 k-mer subsequences and 370 frequent itemset features. The feature calculation may be performed on the original sequence feature set according to the k-mer subsequences of the RNA-protein pairs to obtain a 930-dimensional feature value vector, which may be used as the input of the SVM model to train the model parameters of the model. The model parameters of the SVM model may include a penalty factor C, a gamma parameter, and the like. In the SVM model, the kernel function can be a polynomial kernel function. In order to increase the generalization ability of the model, the penalty term may be reduced and the penalty factor C may be set to 0.8. The radial basis kernel function may also be used as the kernel function, in which the gamma parameter is 1/n_features by default. By adjusting the parameters through experiments, it can be seen that when the gamma parameter value is 0.1, the performance of the model is better and the learning effect is better. After the model parameters are determined, the training data set may be trained by using the model parameters to obtain an SVM model with better performance.

It can be seen from the training of the SVM model that the traditional machine learning model needs to manually design the feature extraction method and set the model parameters, which makes the model interpretability strong, but the generalization ability of the model is weak. Therefore, the CNN model may be used for feature extraction and classification to achieve end-to-end RNA-protein prediction. In addition, the CNN model may be used to extract the feature information of adjacent bases and amino acids, but the relationship between bases and amino acids at a certain interval cannot be extracted. Therefore, the BILSTM model may be used to extract sequence information.

Exemplarily, each RNA-protein pair in the training data set may be vectorized, and the obtained RNA sequence representation vector and the protein sequence representation vector in each RNA-protein pair are input into the CNN model and the BILSTM model. Further, in the following description, the training of the prediction models using the i-th RNA-protein pair is taken as an example.

The sequence features of this RNA-protein pair may be input into the SVM model, and a predicted value y₁ is output. The representation vector of the RNA sequence and the representation vector of the protein sequence in the RNA-protein pair may be input into two CNN sub-models respectively, and the outputs of the two CNN sub-models may be concatenated to finally output a predicted value y₂. Similarly, the representation vector of the RNA-protein pair may be input into the BiLSTM model, and a predicted value y₃ may be output. The output value of each interaction prediction model may be marked as 0 or 1, with 0 indicating that the RNA-protein pair has no interaction and 1 indicating that the RNA-protein pair has an interaction. For the inputs of the CNN model and the BiLSTM model, taking the RNA sequence as an example, the representation vector of the RNA sequence may be the One-Hot vector of the sequence obtained from the One-Hot vectors of multiple bases in the sequence, or representation vector of the RNA sequence may be the 3-mer One-Hot vector of the sequence, or the representation vector of the RNA sequence may be the 4-mer One-Hot vector of the sequence, which is not specifically limited in this disclosure. It can be understood that, for each RNA-protein pair in the training data set, three predicted interaction values can be obtained through the three interaction prediction models.

Table 4 is a network architecture of the CNN model. It can be seen that the RNA sequence and the protein sequence may be input into two CNN sub-networks respectively, and the network architectures of the two CNN sub-networks are the same. Exemplarily, the RNA sequence being used as the input of the first CNN sub-network is taken as an example for description. The first CNN sub-network may contain 5 convolutional layers with a kernel size of 1×3, 4 normalization layers, 4 downsampling layers, a concatenation layer, a fully connected layer, and a dropout layer. Exemplarily, the convolutional layer C1 may use a 1×3 convolution kernel for feature extraction, and the number of output channels is 32. The BN (Batch Normalization) operation may be performed on the sequence features extracted from each channel to characterize the sequence, thereby accelerating network training. The downsampling layer PI may use a 1×2 convolution kernel to take the maximum value of the feature points in the neighborhood to reduce the number of parameters to be learned by the network. The RNA sequence can be down-sampled layer by layer to obtain the feature vector of the RNA sequence. Similarly, the protein sequence can be down-sampled layer by layer to obtain the feature vector of the protein sequence. The concatenation layer of the CNN model may be used to concatenate the feature vector of the RNA sequence and the feature vector of the protein sequence end to end, a bias is added in the fully connected layer to output the concatenated feature vector of the RNA sequence and the feature vector of the protein sequence, and the dropout layer is added to perform training with a neural network that randomly drops a portion of the neural network nodes. In addition, the activation function of the last layer can choose the Sigmoid activation function.

TABLE 4

| | | |
|---|---|---|
| network layer | input sequence 1 | input sequence 2 |
| input | RNA sequence | protein sequence |
| convolutional layer | convolution kernel (1, 3), | convolution kernel (1, 3), |
| C1 | output channels: 32 | output channels: 32 |
| normalization layer | BN operation | BN operation |
| downsampling layer P1 | Max pooling (1, 2) | Max pooling (1, 2) |
| convolutional layer | convolution kernel (1, 3), | convolution kernel (1, 3), |
| C2 | output channels: 32 | output channels: 32 |
| normalization layer | BN operation | BN operation |
| downsampling layer P2 | Max pooling (1, 2) | Max pooling (1, 2) |
| convolutional layer | convolution kernel (1, 3), | convolution kernel (1, 3), |
| C3 | output channels: 16 | output channels: 16 |
| normalization layer | BN operation | BN operation |
| downsampling layer P3 | Max pooling (1, 2) | Max pooling (1, 2) |
| convolutional layer | convolution kernel (1, 3), | convolution kernel (1, 3), |
| C4 | output channels: 16 | output channels: 16 |
| normalization layer | BN operation | BN operation |
| downsampling layer P4 | Max pooling (1, 2) | Max pooling (1, 2) |
| convolutional layer | convolution kernel (1, 1), | convolution kernel (1, 1), |
| C5 | output channel: 1 | output channel: 1 |

| | |
|---|---|
| concatenation layer | concatenation of two feature vectors end to end |

CON

| | |
|---|---|
| fully connected layer | output 32 nodes, with a basis added |

FC1

| | |
|---|---|
| dropout layer | Dropout ratio is 0.2 dropout rate |
| output layer | use Sigmoid activation function, with output of 0/1 |

Table 5 is a network architecture of the BiLSTM model. As can be seen from the table, the RNA sequence and the protein sequence may be input into two identical BiLSTM sub-networks, respectively. Exemplarily, the RNA sequence being be used as the input of the first BiLSTM sub-network is taken as an example for description. If the RNA sequence is “CCAUCUGA”, the representation vector of the RNA sequence may be an 8*4 matrix obtained from the One-Hot vectors of 8 bases in the sequence, or the 3-mer One-Hot vector of the sequence, or the 4-mer One-Hot vector of the sequence. Exemplarily, when the input is an 8*4 matrix obtained from the One-Hot vectors of 8 bases, each base vector may be input into the BiLSTM network in turn to obtain the feature vector of the RNA sequence. The BiLSTM network is the combination of a forward LSTM network and a backward LSTM network. The LSTM network is a time-recurrent neural network, which is suitable for processing and predicting important events with relatively long intervals and delays in time series.

Specifically, the One-Hot vectors of the 8 bases may be input into the forward LSTM network in turn, and a first latent vector of the RNA sequence may be output. At the same time, the One-Hot vectors of the 8 bases may be input into the backward LSTM network in reverse order, and a second latent vector of the RNA sequence may be output. The first latent vector and the second latent vector of the RNA sequence are concatenated to finally obtain the feature vector of the RNA sequence. For example, the One-Hot vector of the first base “C” may be input into the forward LSTM network first, and the latent feature extraction may be performed on the One-Hot vector of “C” through the forward LSTM network, and the latent vector at this time such as time t is output. Then, the latent vector at time t and the One-Hot vector of the second base “C” at time t+1 may be concatenated, and the concatenated vector may be input into the forward LSTM network, and the latent feature extractions is performed on the concatenated vector, and the latent vector at time t+1 is output. Similarly, the One-Hot vector of the base at the current time moment may be concatenated with the latent vector passed down at the previous moment in turn, and the feature extraction of the concatenated vector is carried out through the forward LSTM network, until the One-Hot vector of the eighth base is “A” is input into the forward LSTM network, and the latent vector at the last moment is output, that is, the first latent vector of the RNA sequence is obtained. At the same time, the One-Hot vector of the eighth base “A” may be input into the backward LSTM network first, and the latent feature extraction may be performed on the One-Hot vector of “A” through the backward LSTM network, and the latent vector at the time such as the time t is output. Then, the latent vector at time t may be concatenated with the One-Hot vector of the seventh base “G” at time t+1, and the concatenated vector can be input into the backward LSTM network, and the latent feature extraction may be performed on the concatenated vector, and the latent vector at time t+1 is output. Similarly, the One-Hot vector of the base at the current moment may be concatenated with the latent vector passed down at the previous moment in turn, and the feature extraction of the concatenated vector may be performed through the backward LSTM network, until the One-Hot vector of first base “C” is input into the backward LSTM network, and the latent vector at the last moment is output, that is, the second latent vector of the RNA sequence is obtained. After concatenating the first latent vector and the second latent vector of the RNA sequence, the feature vector of the RNA sequence is obtained. When the feature vector of the RNA sequence is obtained through the BILSTM model, all the forward and backward sequence information of the RNA sequence can be obtained, and the relationship between the bases with a certain interval can also be extracted, so as to predict the interaction between the input RNA sequence and the protein sequence more accurately.

After obtaining the feature vector of the RNA sequence and the feature vector of the protein sequence through the two BiLSTM sub-networks, the feature vector of the RNA sequence and the feature vector of the protein sequence can be concatenated end to end by using the concatenation layer of the BiLSTM model, and the bias is added in the fully connected layer to output the concatenated feature vector of the RNA sequence and the feature vector of the protein sequence, and a dropout layer is added for training using a neural network that randomly drops a portion of the neural network nodes. In addition, the activation function of the last layer can choose the Sigmoid activation function.

| network layer | input sequence 1 | input sequence 2 |
|---|---|---|
| input | RNA sequence | protein sequence |
| BiLSTM | return all column vectors, | return all column vectors, |
| | the number of output channels: 16 | the number of output channels: 16 |
| concatenation layer | concatenation of two feature vectors | |
| fully connected layer | output nodes: 32 | |
| FC1 | | |

| dropout layer | Dropout = 0.5 |
|---|---|
| fully connected layer | output nodes: 16 |
| FC2 | |
| dropout layer | Dropout = 0.2 |
| output layer | Sigmoid activation function, with output of 0/1 |

Exemplarily, when training the CNN model and the BILSTM model, the stochastic gradient descent algorithm may be used to update the model parameters of the two models. According to the principle of back-propagation, the objective function such as the cross-entropy loss function is continuously calculated, and the model parameters of each interaction prediction model are simultaneously updated according to the calculated loss value. When the objective function converges to the minimum value, the training of all model parameters is completed. The model parameters may also be updated iteratively in the reverse direction, and when the preset number of iterations is satisfied, the training of all model parameters is completed. Exemplarily, the preset number of iterations may be 20, and each interaction prediction model is constantly updating model parameters during the 20 reverse iterations. After the iterations are completed, the optimized model parameters can be obtained. In other examples, the objective function can also be minimized by alternating least squares method, Adam optimization algorithm, and so on, and the model parameters can be updated sequentially from the back to the front to optimize the model parameters.

After the training of each model is completed, each final model may be used to predict the interaction between an unknown RNA-protein pair, and multiple predicted values can be obtained, and the multiple predicted values can be fused to obtain the final prediction result. Finally, the predicted result of the interaction of the RNA-protein pair can be output to the terminal device for a user to view.

In an example embodiment of the present disclosure, at least one RNA sequence may be obtained, and at least three interaction prediction models are used to search a database for protein sequences that have interaction with each input RNA sequence. Specifically, after obtaining the original data set, at least three interaction prediction models may be pre-trained by using the original data set with reference to FIG. 5. After training, all protein sequences involved in training may be stored in the database. It should be noted that the database may also include other protein sequences that did not participate in the training, that is, the number of protein sequences in the database may be arbitrary, and the database may include any number of RNA sequences, for example, the database may include but is not limited to all RNA sequences involved in training, and embodiments of the present disclosure do not impose specific limitations on this.

After a user inputs at least one RNA sequence, each input RNA sequence may be combined with all protein sequences in the database into a plurality of RNA-protein pairs. Further, at least three interaction prediction models may be used to predict the interaction of each RNA-protein pair according to steps S220 to S250. Specifically, feature extraction and vectorization may be performed on each RNA-protein pair, and the obtained sequence features, the RNA sequence representation vector and the protein sequence representation vector in the RNA-protein pair are input into at least three interaction prediction models, and the predicted interaction value for each RNA-protein pair is obtained. A predicted interaction value of 1 indicates that the RNA-protein pair has an interaction, and a predicted interaction value of 0 indicates that the RNA-protein pair has no interaction. Then, all RNA-protein pairs with a predicted interaction value of 1 may be selected, and the protein sequence in each RNA-protein pair may be output to the terminal device for the user to view the protein sequences that have interaction with the input RNA sequence.

Similarly, in an example embodiment of the present disclosure, at least one protein sequence may be obtained, and at least one RNA sequence that interacts with each input protein sequence may be searched in the database through at least three interaction prediction models. Exemplarily, after the user inputs at least one protein sequence, each input protein sequence may be combined with all RNA sequences in the database to form a plurality of RNA-protein pairs. Further, the interaction of each RNA-protein pair may be predicted using at least three interaction models according to steps S220 to S250. Specifically, feature extraction and vectorization may be performed on each RNA-protein pair, and the obtained sequence features, the RNA sequence representation vector and the protein sequence representation vector in each RNA-protein pair are input into the at least three interaction prediction models, and the predicted interaction value for each RNA-protein pair is output. A predicted interaction value of 1 indicates that the RNA-protein pair has an interaction, and a predicted interaction value of 0 indicates that the RNA-protein pair has no interaction. Then, all RNA-protein pairs with a predicted interaction value of 1 may be selected, and the RNA sequence in each RNA-protein pair may be output to the terminal device for the user to view the RNA sequences that have interaction with the input protein sequence.

In the RNA-protein interaction prediction method provided by the example embodiment of the present disclosure, the RNA-protein pair to be predicted is obtained; feature extraction is performed on the RNA-protein pair to be predicted to obtain sequence features of the RNA-protein pair to be predicted; the RNA-protein pair to be predicted is vectorized to obtain the RNA sequence representation vector and the protein sequence representation vector in the RNA-protein pair to be predicted; based on the sequence features of the RNA-protein pair to be predicted, the RNA sequence representation vector and the protein sequence representation vector in the RNA-protein pair to be predicted, at least one predicted interaction value of the RNA-protein pair to be predicted is obtained using at least one interaction prediction model; and interaction between the RNA and the protein is determined according to the at least one predicted interaction value.

On the one hand, through feature extraction and vectorization of the RNA-protein pair, the association between the RNA sequence and the protein sequence can be fully mined, so as to accurately predict the interaction between RNA and protein. On the other hand, characteristics of the interaction prediction models may be effectively combined to further improve the accuracy in RNA-protein interaction prediction.

It should be noted that although the various steps of the methods of embodiments of the present disclosure are depicted in the figures in a particular order, this does not require or imply that the steps must be performed in that particular order, or that all illustrated steps must be performed to achieve the desired the results. Additionally or alternatively, certain steps may be omitted, multiple steps may be combined into one step, and/or one step may be decomposed into multiple steps, and the like.

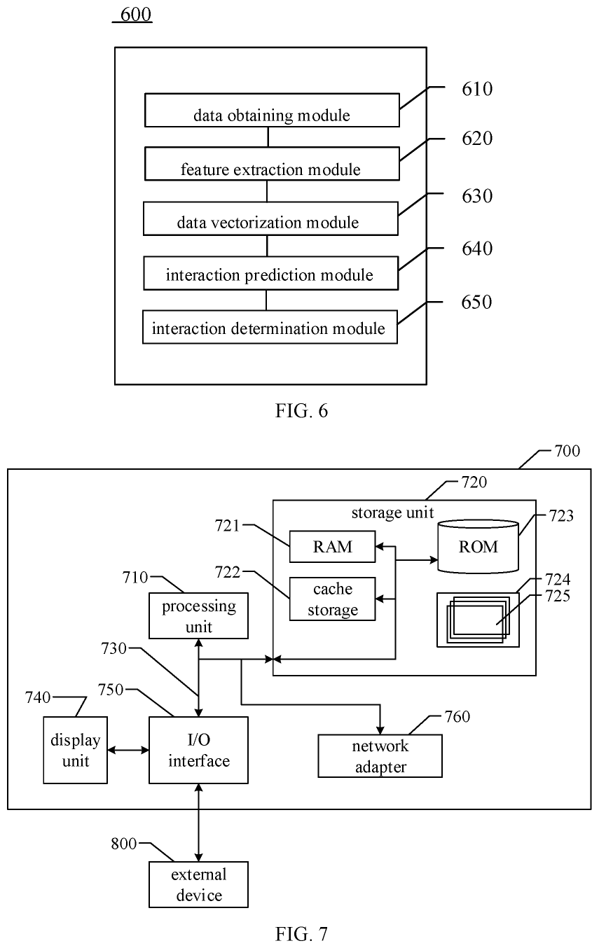

Further, in an example embodiment, an RNA-protein interaction prediction device is also provided. The device may be applied to a server or a terminal device. Referring to FIG. 6, the RNA-protein interaction prediction device 600 may include a data obtaining module 610, a feature extraction module 620, a data vectorization module 630, an interaction prediction module 640 and an interaction determination module 650.

The data obtaining module 610 is configured to obtain an RNA-protein pair to be predicted;
the feature extraction module 620 is configured to perform feature extraction on the RNA-protein pair to be predicted to obtain sequence features of the RNA-protein pair to be predicted;
the data vectorization module 630 is configured to vectorize the RNA-protein pair to be predicted to obtain an RNA sequence representation vector and a protein sequence representation vector in the RNA-protein pair to be predicted;
the interaction prediction module 640 is configured to, based on the sequence features of the RNA-protein pair to be predicted, the RNA sequence representation vector and the protein sequence representation vector in the RNA-protein pair to be predicted, obtain at least one predicted interaction value of the RNA-protein pair to be predicted using at least one interaction prediction model; and
the interaction determination module 650 is configured to determine interaction between the RNA and the protein according to the at least one predicted interaction value.

In an example embodiment, the feature extraction module 620 includes:
a feature set obtain module configured to obtain an original sequence feature set; and
a feature determination module configured to determine the sequence features of the RNA-protein pair to be predicted according to the original sequence feature set.

In an example embodiment, the feature determination module includes:
a sequence conversion unit configured to convert an RNA sequence and a protein sequence in the RNA-protein pair to be predicted into k-mer subsequences, respectively; and
a first sequence search unit configured to search each of the k-mer subsequences in the original sequence feature set, and obtaining the sequence features of the RNA-protein pair to be predicted according to a search result.

In an example embodiment, the feature determination module further includes:
a sequence conversion unit configured to convert an RNA sequence and a protein sequence in the RNA-protein pair to be predicted into k-mer subsequences, respectively, wherein the k-mer subsequences include RNA k-mer subsequences and protein k-mer subsequences;
a sequence combination unit configured to combine the RNA k-mer subsequences and the protein k-mer subsequences to obtain a plurality of RNA-protein k-mer subsequence pairs; and
a second sequence search unit configured to search each of the plurality of RNA-protein k-mer subsequence pairs in the original sequence feature set, and obtaining the sequence features of the RNA-protein pair to be predicted according to a search result.

In an example embodiment, the feature determination module further includes:
a sequence conversion unit configured to convert an RNA sequence and a protein sequence in the RNA-protein pair to be predicted into k-mer subsequences, respectively, wherein the k-mer subsequences include RNA k-mer subsequences and protein k-mer subsequences;
a first sequence search unit configured to search each of the k-mer subsequences in the original sequence feature set to obtain first sequence features;
a sequence combination unit configured to combine the RNA k-mer subsequences and the protein k-mer subsequences to obtain a plurality of RNA-protein k-mer subsequence pairs;
a second sequence search unit configured to search each of the plurality of RNA-protein k-mer subsequence pairs in the original sequence feature set to obtain second sequence features; and
a feature concatenation unit configured to constitute the sequence features of the RNA-protein pair to be predicted by the first sequence features and the second sequence features.

In an example embodiment, the data vectorization module 630 includes:
a sequence conversion unit configured to convert an RNA sequence and a protein sequence in the RNA-protein pair to be predicted into k-mer subsequences, respectively, wherein the k-mer subsequences include M RNA k-mer subsequences and N protein k-mer subsequences;
a first vectorization unit configured to vectorize each of the RNA k-mer subsequences to obtain M RNA k-mer vectors;
a first concatenation unit configured to concatenate the M RNA k-mer vectors to obtain the RNA sequence representation vector;
a second vectorization unit configured to vectorize each of the protein k-mer sequences to obtain N protein k-mer vectors; and
a second concatenation unit configured to concatenate the N protein k-mer vectors to obtain the protein sequence representation vector.

In an example embodiment, the data vectorization module 630 includes:
a base vector obtaining unit configured to vectorize each base included in an RNA sequence in the RNA-protein pair to be predicted to obtain a plurality of base vectors;
a base vector concatenation unit configured to concatenate the plurality of base vectors to obtain the RNA sequence representation vector;
an amino acid vector obtaining unit configured to vectorize each amino acid vector included in a protein sequence in the RNA-protein pair to be predicted to obtain a plurality of amino acid vectors; and
an amino acid vector concatenation unit configured to concatenate the plurality of amino acid vectors to obtain the protein sequence representation vector.

In an example embodiment, the interaction prediction module 640 configured to, based on the sequence features of the RNA-protein pair to be predicted, the RNA sequence representation vector and the protein sequence representation vector in the RNA-protein pair to be predicted, obtain a plurality of predicted interaction values of the RNA-protein pair to be predicted using at least three interaction prediction models.

In an example embodiment, the interaction prediction module 640 includes:
a first prediction unit configured to input the sequence features of the RNA-protein pair to be predicted into a first interaction prediction model to obtain a first predicted interaction value; and
a second prediction unit configured to input the RNA sequence representation vector and the protein sequence representation vector in the RNA-protein pair to be predicted into a second interaction prediction model to obtain a second predicted interaction value;
wherein at least one first interaction model and at least two second interaction prediction models are included; or, at least two first interaction models and at least one second interaction prediction model are included.

In an example embodiment, the interaction prediction module 640 further includes:
a third prediction unit configured to input the sequence features of the RNA-protein pair to be predicted into a traditional machine learning model to obtain a first predicted interaction value; and
a fourth prediction unit configured to input the RNA sequence representation vector and the protein sequence representation vector in the RNA-protein pair to be predicted into a deep learning model to obtain a second predicted interaction value;
wherein at least one traditional machine learning model and at least two deep learning models are included; or, at least two traditional machine learning models and at least one deep learning models are included.

In an example embodiment, the traditional machine learning model includes at least one of a support vector machine model, a logistic regression model and a decision tree model, and the deep learning model includes at least one of a convolutional neural network model and a recurrent neural network model.

In an example embodiment, the interaction determination module 650 includes:
a predicted value marking unit configured to mark a plurality of predicated interaction values to obtain a plurality of marker values; and
an interaction determination unit configured to sum the plurality of marker values, and determining the interaction between the RNA and the protein according to a sum result.

In an example embodiment, the feature set obtaining module includes:
a data set obtaining module configured to obtain an original data set;
a feature extraction module configured to perform feature extraction on each RNA-protein pair in the original data set to obtain the original sequence feature set.

In an example embodiment, the feature extraction module includes:
a sequence generation unit configured to perform permutation and combination on basic units of the RNA and the protein respectively to obtain k-mer subsequences;
a variance calculation unit configured to calculate an average value of the number of occurrences of each k-mer subsequence in each RNA-protein pair, and calculate a variance of each k-mer subsequence according to the average value of the number of occurrences; and
a data determination unit configured to determine the original sequence feature set according to a magnitude of the variance of each k-mer subsequence.

In an example embodiment, the variance calculation unit includes:
a number counting subunit configured to traverse the original data set to determine the number of occurrences of each k-mer subsequence in each RNA-protein pair;
a total number counting subunit configured to count the number of occurrences of each k-mer subsequence in each RNA-protein pair to obtain a total number of occurrences of each k-mer subsequence in the original data set;
an average number determination subunit configured to calculate the average value of the number of occurrences of each K-mer subsequence in each RNA-protein pair according to the total number of occurrences; and
a variance calculation subunit configured to calculate the variance of each k-mer subsequence according to the average value of the number of occurrences of each k-mer subsequence in each RNA-protein pair and the number of occurrences of each k-mer subsequence in each RNA-protein pair.

In an example embodiment, the variance calculation subunit is configured to calculate the variance s² of each k-mer subsequence according to:

$${s}^{2} = \frac{{( m - {x}_{1} )}^{2} + {( m - {x}_{2} )}^{2} + … + {( m - {x}_{n} )}^{2}}{n}$$

where n is the number of RNA-protein pairs in the original data set, m is the average value of the number of occurrences of each k-mer subsequence in each RNA-protein pair, and xₙ is the number of occurrence of each k-mer subsequence in an n-th RNA-protein pair.

In an example embodiment, the data set determination unit is configured to determine k-mer subsequences which meet a preset condition according to the variance of each k-mer subsequence, and constituting the original sequence feature set by the k-mer subsequences which meet the preset condition.

In an example embodiment, the feature extraction module further includes:
a subsequence pair generation unit configured to convert an RNA sequence and a protein sequence in each RNA-protein pair into k-mer subsequences respectively to obtain k-mer subsequence pairs; and
a feature set obtaining unit configured to count a relative frequency of occurrence of each of the k-mer subsequence pairs in the original data set, and constituting the original sequence feature set by k-mer subsequence pairs which meet a preset condition for relative frequency of occurrence.

In an example embodiment, the RNA-protein interaction prediction device 600 further includes:
a training module configured to train the at least one interaction prediction model.

In an example embodiment, the training module includes:
a training data obtaining unit configured to obtain a training data set, wherein the training data set includes positive RNA-protein pairs and negative RNA-protein pairs;
a model parameter training unit configured to, with the training data set as an input of the at least one interaction prediction model, iteratively update model parameters of the at least one interaction prediction model, and when an iteration termination condition is met, completing training of all model parameters so as to predict the interaction of the RNA-protein pair to be predicted using the trained at least one interaction prediction model.

In an example embodiment, the RNA-protein interaction prediction device 600 further includes:

A data output module configured to output a prediction result of the interaction between the RNA and the protein.

The specific details of each module in the above-mentioned RNA-protein interaction prediction device have been described in detail in the corresponding RNA-protein interaction prediction methods, and thus repeated descriptions will be omitted here.

Each module in the above device may be a general-purpose processor, including: a central processing unit, a network processor, and so on; it can also be a digital signal processor, an application-specific integrated circuit, a field programmable gate array or other programmable logic devices, a discrete gate or a transistor logic device, a discrete hardware component. Each module can also be implemented in the form of software, firmware and the like. Each processor in the above device may be an independent processor, or may be integrated together.

An example embodiment of the present disclosure also provides a computer-readable storage medium having stored thereon a program product capable of implementing the above methods according to embodiments of the present disclosure. In some possible implementations, aspects of the present disclosure may also be implemented in the form of a program product, which includes program codes. When the program product is run on an electronic device, the program codes are used to cause the electronic device to perform the steps according to various example embodiments of the present disclosure described in the above-mentioned exemplary methods.

The program product may be stored by a portable compact disc read-only memory (CD-ROM) and include program codes, and may be executed on a terminal device, such as a personal computer. However, the program product of the present disclosure is not limited thereto. The readable storage medium may be any tangible medium containing or storing a program, and the program may be used by an instruction execution system, apparatus, or device, or the program may be used in combination with an instruction execution system, apparatus, or device.

The program product may employ any combination of one or more readable mediums. The readable medium may be a readable signal medium or a readable storage medium. The readable storage medium may be, for example, but is not limited to, an electronic, magnetic, optical, electromagnetic, infrared, or semiconductor system, apparatus, or device, or any combination thereof. More specific examples (non-exhaustive examples) of readable storage media include: electrical connection with one or more wires, portable disk, hard disk, random access memory (RAM), read-only memory (ROM), erasable programmable read-only memory (EPROM or flash memory), optical fiber, portable compact disc read-only memory (CD-ROM), optical storage device, magnetic storage device, or any suitable combination of the foregoing.

The computer-readable signal medium may include a data signal in baseband or propagated as part of a carrier wave, which carries readable program codes. Such a propagated data signal may have many forms, including but not limited to electromagnetic signals, optical signals, or any suitable combination of the foregoing. The readable signal medium may also be any readable medium other than a readable storage medium, and the readable medium may send, propagate, or transmit a program that is used by an instruction execution system, apparatus, or device, or that is used in combination with an instruction execution system, apparatus, or device.

The program codes contained on the readable medium may be transmitted using any appropriate medium, including but not limited to wireless, wired, optical fiber, RF, etc., or any suitable combination of the foregoing.

The program codes for performing the operations of the present disclosure can be written in any combination of one or more programming languages, which include object-oriented programming languages, such as Java, C++, and so on. The programming languages also include conventional procedural programming language, such as “C” or a similar programming language. The program codes can be executed entirely on the user computing device, can be executed partly on the user device, can be executed as an independent software package, can be executed partly on the user computing device and partly on a remote computing device, or can be executed entirely on the remote computing device or server. In the case of a remote computing device, the remote computing device can be connected to the user computing device through any kind of network, including a local area network (LAN) or a wide area network (WAN), or the remote computing device can be connected to an external computing device, for example, by the Internet provided by the Internet service providers.

An example embodiment of the present disclosure also provides an electronic device capable of implementing the above methods. An electronic device 700 according to this example embodiment of the present disclosure is described below with reference to FIG. 7. The electronic device 700 shown in FIG. 7 is only an example, and should not impose any limitation on the functions and scope of use of the embodiments of the present disclosure.

As shown in FIG. 7, the electronic device 700 is shown in the form of a general-purpose computing device. The components of the electronic device 700 may include, but are not limited to, at least one processing unit 710, at least one storage unit 720, and a bus 730 connecting different system components (including the storage unit 720 and the processing unit 710), and a display unit 740.

The storage unit stores program codes, and the program codes can be executed by the processing unit 710, so that the processing unit 710 executes various example embodiments according to the present disclosure described in the “exemplary methods” section of the present specification. For example, the processing unit 710 may perform any one or more of the steps shown in FIG. 2 to FIG. 7.

The storage unit 720 may include a readable medium in the form of a volatile storage unit, such as a random access storage unit (RAM) 721 and/or a cache storage unit 722, and may further include a read-only storage unit (ROM) 723.

The storage unit 720 may further include a program/utility tool 724 having a set (at least one) of program modules 725. Such program modules 725 include, but are not limited to, an operating system, one or more application programs, other program modules, and program data. Each or some combination of these examples may include an implementation of a network environment.

The bus 730 may be one or more of several types of bus structures, including a memory unit bus or a memory unit controller, a peripheral bus, a graphics acceleration port, a processing unit, or a local area bus using any bus structure in a variety of bus structures.

The electronic device 700 may also communicate with one or more external devices 800 (such as a keyboard, a pointing device, a Bluetooth device, etc.), and may also communicate with one or more devices that enable a user to interact with the electronic device 700, and/or may also communicate with any device (such as a router, a modem) that can enable the electronic device 700 to interact with one or more other computing devices. Such communication can be performed through an input/output (I/O) interface 750. Moreover, the electronic device 700 may also communicate with one or more networks (such as a local area network (LAN), a wide area network (WAN), and/or a public network, such as the Internet) through the network adapter 760. As shown in the figure, the network adapter 760 communicates with other modules of the electronic device 700 through the bus 730. It should be understood that although not shown in the figure, other hardware and/or software modules may be used in conjunction with the electronic device 700, including but not limited to: microcode, device drivers, redundant processing units, external disk drive arrays, RAID systems, tape drives and data backup storage systems.

In some embodiments, the processing unit 710 of the electronic device may be configured to perform the RNA-protein interaction prediction method in embodiments of the present disclosure. In some embodiments, the RNA-protein pair to be predicted/RNA sequence to be predicted/protein sequence to be predicted, the original data set, and the training data set for training each interaction prediction model, and so on, can be input through the input interface 750. For example, the RNA-protein pair to be predicted, the original data set, and the training data set for training each interaction prediction model and so on are input through the user interface of the electronic device. In some embodiments, the prediction result of the interaction of the RNA-protein pair to be predicted may be output to the external device 800 through the output interface 750 for the user to view.

Through the description of the foregoing embodiments, those skilled in the art can easily understand that the example embodiments described herein can be implemented by software, or by software in combination with necessary hardware. Therefore, the technical solutions according to the embodiments of the present disclosure may be embodied in the form of a software product, and the software product may be stored in a non-volatile storage medium (which may be a CD-ROM, a U disk, a mobile hard disk, etc.) or on a network. The software product may include instructions to cause a computing device (which may be a personal computer, a server, a terminal device, or a network device, etc.) to execute the methods according to exemplary embodiments of the present disclosure.

In addition, the drawings are merely schematic descriptions of processes included in the methods according to example embodiments of the present disclosure, and are not for limiting the present disclosure. It is easy to understand that the processes shown in the drawings do not indicate or limit the chronological order of these processes. In addition, it is also easy to understand that these processes may be performed synchronously or asynchronously in multiple modules, for example.

It should be noted that although several modules or units of the devices for action execution are described above, such division is not mandatory. In fact, according to the embodiments of the present disclosure, the features and functions of two or more modules or units described above may be embodied in one module or unit. Conversely, the features and functions of a module or unit described above can be further divided into multiple modules or units. It is to be understood that the present disclosure is not limited to the precise structures described above and illustrated in the accompanying drawings, and that various modifications and changes may be made without departing from the scope of the present disclosure. The scope of the present disclosure is limited only by the appended claims.

1. An RNA-protein interaction prediction method, comprising:
obtaining an RNA-protein pair to be predicted;
performing feature extraction on the RNA-protein pair to be predicted to obtain sequence features of the RNA-protein pair to be predicted;
vectorizing the RNA-protein pair to be predicted to obtain an RNA sequence representation vector and a protein sequence representation vector in the RNA-protein pair to be predicted;
based on the sequence features of the RNA-protein pair to be predicted, the RNA sequence representation vector and the protein sequence representation vector in the RNA-protein pair to be predicted, obtaining at least one predicted interaction value of the RNA-protein pair to be predicted using at least one interaction prediction model; and
determining interaction between the RNA and the protein according to the at least one predicted interaction value.

2. The RNA-protein interaction prediction method according to claim 1, wherein performing the feature extraction on the RNA-protein pair to be predicted to obtain the sequence features of the RNA-protein pair to be predicted comprises:
obtaining an original sequence feature set; and
determining the sequence features of the RNA-protein pair to be predicted according to the original sequence feature set.

3. The RNA-protein interaction prediction method according to claim 2, wherein determining the sequence features of the RNA-protein pair to be predicted according to the original sequence feature set comprises:
converting an RNA sequence and a protein sequence in the RNA-protein pair to be predicted into k-mer subsequences, respectively; and
searching each of the k-mer subsequences in the original sequence feature set, and obtaining the sequence features of the RNA-protein pair to be predicted according to a search result.

4. The RNA-protein interaction prediction method according to claim 2, wherein determining the sequence features of the RNA-protein pair to be predicted according to the original sequence feature set comprises:
converting an RNA sequence and a protein sequence in the RNA-protein pair to be predicted into k-mer subsequences, respectively, wherein the k-mer subsequences comprise RNA k-mer subsequences and protein k-mer subsequences;
combining the RNA k-mer subsequences and the protein k-mer subsequences to obtain a plurality of RNA-protein k-mer subsequence pairs; and
searching each of the plurality of RNA-protein k-mer subsequence pairs in the original sequence feature set, and obtaining the sequence features of the RNA-protein pair to be predicted according to a search result.

5. The RNA-protein interaction prediction method according to claim 2, wherein determining the sequence features of the RNA-protein pair to be predicted according to the original sequence feature set comprises:
converting an RNA sequence and a protein sequence in the RNA-protein pair to be predicted into k-mer subsequences, respectively, wherein the k-mer subsequences comprise RNA k-mer subsequences and protein k-mer subsequences;
searching each of the k-mer subsequences in the original sequence feature set to obtain first sequence features;
combining the RNA k-mer subsequences and the protein k-mer subsequences to obtain a plurality of RNA-protein k-mer subsequence pairs;
searching each of the plurality of RNA-protein k-mer subsequence pairs in the original sequence feature set to obtain second sequence features; and
constituting the sequence features of the RNA-protein pair to be predicted by the first sequence features and the second sequence features.

6. The RNA-protein interaction prediction method according to claim 1, wherein vectorizing the RNA-protein pair to be predicted to obtain the RNA sequence representation vector and the protein sequence representation vector in the RNA-protein pair to be predicted comprises:
converting an RNA sequence and a protein sequence in the RNA-protein pair to be predicted into k-mer subsequences, respectively, wherein the k-mer subsequences comprise M RNA k-mer subsequences and N protein k-mer subsequences;
vectorizing each of the RNA k-mer subsequences to obtain M RNA k-mer vectors;
concatenating the M RNA k-mer vectors to obtain the RNA sequence representation vector;
vectorizing each of the protein k-mer subsequences to obtain N protein k-mer vectors; and
concatenating the N protein k-mer vectors to obtain the protein sequence representation vector.

7. The RNA-protein interaction prediction method according to claim 1, wherein vectorizing the RNA-protein pair to be predicted to obtain the RNA sequence representation vector and the protein sequence representation vector in the RNA-protein pair to be predicted comprises:
vectorizing each base comprised in an RNA sequence in the RNA-protein pair to be predicted to obtain a plurality of base vectors;
concatenating the plurality of base vectors to obtain the RNA sequence representation vector;
vectorizing each amino acid vector comprised in a protein sequence in the RNA-protein pair to be predicted to obtain a plurality of amino acid vectors; and
concatenating the plurality of amino acid vectors to obtain the protein sequence representation vector.

8. The RNA-protein interaction prediction method according to claim 1, wherein based on the sequence features of the RNA-protein pair to be predicted, the RNA sequence representation vector and the protein sequence representation vector in the RNA-protein pair to be predicted, obtaining the at least one predicted interaction value of the RNA-protein pair to be predicted using the at least one interaction prediction model comprises:
based on the sequence features of the RNA-protein pair to be predicted, the RNA sequence representation vector and the protein sequence representation vector in the RNA-protein pair to be predicted, obtaining a plurality of predicted interaction values of the RNA-protein pair to be predicted using at least three interaction prediction models.

9. The RNA-protein interaction prediction method according to claim 1, wherein based on the sequence features of the RNA-protein pair to be predicted, the RNA sequence representation vector and the protein sequence representation vector in the RNA-protein pair to be predicted, obtaining the at least one predicted interaction value of the RNA-protein pair to be predicted using the at least one interaction prediction model comprises:
inputting the sequence features of the RNA-protein pair to be predicted into a first interaction prediction model to obtain a first predicted interaction value; and
inputting the RNA sequence representation vector and the protein sequence representation vector in the RNA-protein pair to be predicted into a second interaction prediction model to obtain a second predicted interaction value;
wherein at least one first interaction model and at least two second interaction prediction models are comprised; or, at least two first interaction models and at least one second interaction prediction model are comprised.

10. The RNA-protein interaction prediction method according to claim 1, wherein based on the sequence features of the RNA-protein pair to be predicted, the RNA sequence representation vector and the protein sequence representation vector in the RNA-protein pair to be predicted, obtaining the at least one predicted interaction value of the RNA-protein pair to be predicted using the at least one interaction prediction model comprises:
inputting the sequence features of the RNA-protein pair to be predicted into a traditional machine learning model to obtain a first predicted interaction value; and
inputting the RNA sequence representation vector and the protein sequence representation vector in the RNA-protein pair to be predicted into a deep learning model to obtain a second predicted interaction value;
wherein at least one traditional machine learning model and at least two deep learning models are comprised; or, at least two traditional machine learning models and at least one deep learning models are comprised.

11. The RNA-protein interaction prediction method according to claim 10, wherein the traditional machine learning model comprises at least one of a support vector machine model, a logistic regression model and a decision tree model, and the deep learning model comprises at least one of a convolutional neural network model and a recurrent neural network model.

12. The RNA-protein interaction prediction method according to claim 1, wherein the at least one predicted value comprises a plurality of predicted values;
wherein determining the interaction between the RNA and the protein according to the at least one predicted interaction value comprises:
marking the plurality of predicated interaction values to obtain a plurality of marker values; and
summing the plurality of marker values, and determining the interaction between the RNA and the protein according to a sum result.

13. The RNA-protein interaction prediction method according to claim 2, wherein obtaining the original sequence feature set comprises:
obtaining an original data set;
performing feature extraction on each RNA-protein pair in the original data set to obtain the original sequence feature set.

14. The RNA-protein interaction prediction method according to claim 13, wherein performing the feature extraction on each RNA-protein pair in the original data set to obtain the original sequence feature set comprises:
performing permutation and combination on basic units of the RNA and the protein respectively to obtain k-mer subsequences;
calculating an average value of the number of occurrences of each k-mer subsequence in each RNA-protein pair, and calculating a variance of each k-mer subsequence according to the average value of the number of occurrences; and
determining the original sequence feature set according to a magnitude of the variance of each k-mer subsequence.

15. The RNA-protein interaction prediction method according to claim 14, wherein calculating the average value of the number of occurrences of each k-mer subsequence in each RNA-protein pair, and calculating the variance of each k-mer subsequence according to the average value of the number of occurrences comprises:
traversing the original data set to determine the number of occurrences of each k-mer subsequence in each RNA-protein pair;
counting the number of occurrences of each k-mer subsequence in each RNA-protein pair to obtain a total number of occurrences of each k-mer subsequence in the original data set;
calculating the average value of the number of occurrences of each K-mer subsequence in each RNA-protein pair according to the total number of occurrences; and
calculating the variance of each k-mer subsequence according to the average value of the number of occurrences of each k-mer subsequence in each RNA-protein pair and the number of occurrences of each k-mer subsequence in each RNA-protein pair.

16. The RNA-protein interaction prediction method according to claim 15, wherein calculating the variance of each k-mer subsequence according to the average value of the number of occurrences of each k-mer subsequence in each RNA-protein pair and the number of occurrences of each k-mer subsequence in each RNA-protein pair comprises:
calculating the variance s² of each k-mer subsequence according to:
$${s}^{2} = \frac{{( m - {x}_{1} )}^{2} + {( m - {x}_{2} )}^{2} + … + {( m - {x}_{n} )}^{2}}{n}$$
where n is the number of RNA-protein pairs in the original data set, m is the average value of the number of occurrences of each k-mer subsequence in each RNA-protein pair, and xₙ is the number of occurrence of each k-mer subsequence in an n-th RNA-protein pair.

17. The RNA-protein interaction prediction method according to claim 14, wherein determining the original sequence feature set according to the magnitude of the variance of each k-mer subsequence comprises:
determining k-mer subsequences which meet a preset condition according to the variance of each k-mer subsequence, and constituting the original sequence feature set by the k-mer subsequences which meet the preset condition.

18. The RNA-protein interaction prediction method according to claim 13, wherein performing feature extraction on each RNA-protein pair in the original data set to obtain the original sequence feature set comprises:
converting an RNA sequence and a protein sequence in each RNA-protein pair into k-mer subsequences respectively to obtain k-mer subsequence pairs; and
counting a relative frequency of occurrence of each of the k-mer subsequence pairs in the original data set, and constituting the original sequence feature set by k-mer subsequence pairs which meet a preset condition for relative frequency of occurrence.

19. (canceled)

20. (canceled)

21. The RNA-protein interaction prediction method according to claim 1, further comprising:
outputting a prediction result of the interaction between the RNA and the protein.

22. (canceled)

23. (canceled)

24. An electronic device, comprising:
a processor; and
a memory for storing executable instructions which are executable by the processor;
wherein when the executable instructions are executed by the processor, the processor is configured to:
obtain an RNA-protein pair to be predicted;
perform feature extraction on the RNA-protein pair to be predicted to obtain sequence features of the RNA-protein pair to be predicted;
vectorize the RNA-protein pair to be predicted to obtain an RNA sequence representation vector and a protein sequence representation vector in the RNA-protein pair to be predicted;
based on the sequence features of the RNA-protein pair to be predicted, the RNA sequence representation vector and the protein sequence representation vector in the RNA-protein pair to be predicted, obtain at least one predicted interaction value of the RNA-protein pair to be predicted using at least one interaction prediction model; and
determine interaction between the RNA and the protein according to the at least one predicted interaction value.
